(19) United States
(12) Patent Application Publication
(10) Pub. No.: US 2025/0277274 A1
(43) Pub. Date: Sep. 4, 2025

(54) MICROBIOME SIGNATURE

(71) Applicant: Givaudan SA, VERNIER (CH)

(72) Inventors: Catherine ZANCHETTA, Toulouse (FR); David VILANOVA, Escaldes (AD); Cyrille JARRIN, Muret (FR)

(21) Appl. No.: 18/031,068

(22) PCT Filed: Oct. 12, 2021
(86) PCT No.: PCT/EP2021/078111

§ 371 Date: Apr. 10, 2023

(30) Foreign Application Priority Data
Oct. 13, 2020 (GB) 2016237.6

(51) Int. Cl. C12Q 1/689; C12Q 1/6874

(52) U.S. Cl. CPC C12Q 1/689; C12Q 1/6874

(57) ABSTRACT

The present invention relates to methods of identifying microbial signatures that are predictive of particular skin characteristics, and methods of using the signatures to predict skin characteristics.

### FIELD OF THE INVENTION

The invention is in the field of cosmetic skin treatments and methods of improving the quality of the skin.

### BACKGROUND

The skin microbiota represents the living microorganisms on the surface of the skin. In the wake of numerous studies devoted to the intestinal microbiota and demonstration of its major contribution to human health, study of the cutaneous or skin microbiota has grown considerably over the last years. Curing skin diseases such as acne, atopic dermatitis or psoriasis has long been a driving force in investigating the skin microbiota.

Today, awareness of the significance of the skin microbiota greatly exceeds the sphere of pathogenic skin microbial ecology, and understanding of the complex interactions between healthy human skin and the microorganisms inhabiting it promises major developments in the field of dermocosmetics. Thus, there is an increasing interest in cosmetic products that are able to preserve the natural microbial balance of healthy and beautiful skin.

Bacteria, fungi, viruses and archaea are the main microorganisms constituting the microbiota, which is defined as the microbial communities inhabiting a specific environment. In humans, many efforts have been made to characterize the different body site ecosystems and their associated microbial communities, mainly at bacterial level, which are the most abundant microorganisms on the human-associated microbiota. In addition to different skin sites being characterised by different ecosystems, different skin types have a characteristic microbial profile. For example Jarrin et al 2020 (1) IFSCC 45-54 show a microbial profile characteristic of a sensitive-skin phenotype rather than a non-sensitive-skin phenotype.

However, there is a need to better characterise both sensitive skin, and other skin types so that appropriate cosmetic treatments may be advised and used.

### BRIEF SUMMARY OF THE INVENTION

The inventors have surprisingly identified a means for reliably generating a skin microbial signature that can predict whether a subject has skin that has a high level of a particular characteristic, such as hydration or sebum, a low level of that characteristic, or an intermediate level of the characteristic. This means can be used to determine microbial signatures for any skin characteristic, and is exemplified herein with respect to sebum, hydration, age, brown spots, sensitivity, and skin barrier function.

The invention also provides a range of microbial signatures that can be used to predict if a subject has a skin type that falls into any one or more of the above characteristics, or can be used to determine all of the skin characteristics. The method also provides various kits and devices for putting any of the methods of the invention into practice, along with various compositions comprising the microbes associated with particular microbial signatures for cosmetic use.

### DETAILED DESCRIPTION OF THE INVENTION

As described herein, the inventors have found that it is possible to predict whether a subject has skin that has any one or more particular characteristic, for example one or more cosmetic characteristics, or one or more medical characteristics, by identifying the microbial profile of the skin and comparing it to one or more microbial signatures that are predictive of a high level of the characteristic, a low level of the characteristic, or an intermediate level of the characteristic. In a preferred embodiment the characteristic is a cosmetic characteristic. However, the skilled person will appreciate that the premise underlying the invention, being that different types of skin are populated with a different microbiome, can also apply to medical conditions such as eczema versus non-eczema skin. Accordingly, although the present disclosure focuses on the cosmetic embodiments, it should be interpreted as applying equally to medical skin characteristics. To make an accurate prediction, it is important to understand the microbial profile of skin that has a high level and a low level of the particular characteristic, and identify a particular “signature” that indicates a high level or a low level of the characteristic. This signature may be the presence or absence of one or more microbial species or genera, or may be the abundance or relative abundance of one or more microbial species or genera. For example it may not simply be the presence of genera A and B that indicates a high level of the characteristic rather than a low level, but could be the relative ratio of those genera that indicate a high level of the characteristic versus those genera that indicate a low level of the characteristic.

The invention provides a method of identifying a microbial signature predictive of either a high level of a first characteristic or a low level of the same first characteristic wherein the method comprises:
a) (i) providing a number of control skin microbial samples wherein the samples are taken from more than one subject showing a high level of the characteristic, generating a microbial profile from the skin microbial samples, and optionally calculating the percentage abundance of each genera in each sample
b) (ii) providing a number of control skin microbial samples wherein the samples are taken from more than one subject showing a low level of the characteristic, generating a microbial profile from the skin microbial samples, and optionally calculating the percentage abundance of each genera in each sample optionally
c) generating a mean percentage abundance of each genera from the samples showing a high level of the characteristic
d) generating a mean percentage abundance of each genera from the samples showing a low level of the characteristic
e) identifying genera of microbes that are more prevalent in skin showing the high level of the characteristic
f) identifying genera of microbes that are more prevalent in skin showing the low level of the characteristic and optionally
g) i) for the control skin samples showing a high level of the characteristic, summing the mean percentage abundance of each genera identified in (e) and summing the mean percentage abundance of each genera identified in (f); and
   ii) calculating the ratio of the sum of the mean percentage abundance of each genera identified in (e) to the sum of the mean percentage abundance of each genera identified in (f) giving an upper threshold ratio; and
iii) for the control skin samples showing a low level of the characteristic, summing the mean percentage abundance of each genera identified in (e) and summing the mean percentage abundance of each genera identified in (f); and
iv) calculating the ratio of the sum of the mean percentage abundance of each genera identified in (e) to the sum of the mean percentage abundance of each genera identified in (f) giving a lower threshold
wherein:
   a test sample having a test ratio of greater than the upper threshold ratio is considered to be a sample that has a high level of the characteristic;
   a test sample having a test ratio of lower than the lower threshold ratio is considered to be a sample that has a low level of the characteristic; and
   a test sample having a test ratio of between the upper threshold ratio and the lower threshold ratio is considered to be a sample that has an intermediate level of the characteristic; and/or
h) wherein a test sample comprising genera of microbes that are more prevalent in skin showing a high level of the characteristic is considered to predict that the skin has a high level of the characteristic, and wherein a test sample comprising genera of microbes that are more prevalent in skin showing a low level of the characteristic is considered to predict that the skin has a low level of the characteristic.

The method of the present invention may be performed on human skin microbial samples obtained from any desired area of the body, not only from the face, but also from the scalp, axilla or forearm.

The human skin sample may be obtained by any suitable method, for instance by swabbing, scrubbing or scraping. Non-invasive sampling methods are generally preferred. In preferred embodiments, the skin microbial samples have been obtained by swabbing the skin, preferably swabbing skin from the face of the subject, for example from the cheek, or forehead.

In an embodiment, the human skin sample is obtained by swabbing. For instance, swabbing may be performed using sterile swabs (e.g. Medicomp, Hartmann; or forensic swabs from Sarstadt; swabs may be made of viscose, optionally also containing some polyester, e.g. 100% viscose or 70% viscose+30% polyester), which are preferably moistened with physiological serum (e.g. serum physiologique, Mercurochrome), with a sterile NaCl solution (e.g. 0.15 M NaCl), or mixtures thereof. Moisturizing the swab prior to swabbing helps to avoid abrasion of the skin.

The aim of the method is to generate a profile that is representative of subjects with a high level and a low level of the characteristic. This requires at least two samples to be used from a subject with a high level of the characteristic, and at least two samples to be used from a subject with a low level of the characteristic. These samples can be considered to be control samples-they are taken from subjects that are known to have either a low or a high level of the characteristic. The skilled person appreciates that for such methods the statistical power is greater the larger the sample size. Accordingly in one embodiment the number of samples used from a subject with a high level of the characteristic is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 or more; and the number of samples used from a subject with a low level of the characteristic is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 or more.

It is preferred if the only variable between all of the subjects is the level of the characteristic under analysis. For example, in one embodiment the subjects are assessed for a number of characteristics and are only selected for inclusion in the method if they are matched for all conditions other than the one under investigation (see for example Table 1).

The microbial profile can be generated by any available means. For example in one embodiment nucleic acid is extracted from microbes present in the sample and analysis is performed on the nucleic acid, for example the nucleic acid may be amplified and then sequenced to identify the microbes. In some embodiments it may not be necessary to extract the nucleic acid, and amplification and sequencing can be performed on a crude lysate of the microbial sample.

Alternatively, the microbial profile may involve culturing the microbes present in the sample. A disadvantage of this method is that some microbes are not readily culturable. However, of those that are culturable it is still considered possible to identify differences in the profile of the culturable microorganisms between skin having a high or a low level of the characteristic. In another embodiment, other method such as antibody based detection methods can be used.

Preferably, nucleic acid is extracted and the microbes are identified by nucleic acid amplification and sequencing.

Methods of nucleic acid extraction, amplification and sequencing are known and suitable methods can be selected by the skilled person. For example, depending on the type of microbe that is being quantified, the skilled person can select the appropriate primers and cycle conditions so that appropriate regions of the nucleic acid are amplified and sequenced. PCT application PCT/EP20201075530 provides details of appropriate methods (which is hereby incorporated by reference). In one embodiment described herein, DNA is extracted from the sample using the DNeasy PowerLyzer PowerSoil DNA Isolation Kit (Qiagen, Germantown, MD, USA) with the following modifications. The tip of each swab is detached with a sterile surgical blade and transferred to a 1.5 ml tube containing 750 μL of Bead Solution. The sampled biomass is suspended by stirring and pipetting and then transferred to a bead beating tube. The remaining steps can be performed according to the manufacturer's instructions. The DNA concentration was determined using the QuBit dsDNA HS fluorometric quantitation kit (Invitrogen, ThermoFisher Scientific, Courtaboeuf, France) according to the manufacturer's instructions.

In one embodiment, a whole genome sequencing approach is used (e.g. using next-generation sequencing e.g. Sanger-sequencing).

Preferably a more targeted approach is used. It is common in taxonomy to simply amplify a particular region of the genome which is considered to be unique to every species and sequence only that region for comparison with database information. The most common strategy to assess bacterial microbiota is amplifying and sequencing specific regions of 16S rRNA gene using 2^{nd} generation massive sequencing technologies. This bacterial marker gene is ubiquitously found in bacteria, and has nine hypervariable regions (V1-V9) that can be used to infer taxonomy.

Accordingly in one embodiment the method comprises amplifying and sequencing, or simply sequencing, a region of the 16S rRNA bacterial gene; in one embodiment the method comprises amplifying and sequencing, or simply sequencing, any one or more of the V1, V2, V3, V4, V5, V6, V7 or V9 region of the 16S rRNA bacterial gene.

In one embodiment DNA amplification and sequencing of the V3 and V4 16S rRNA gene is performed.

In one embodiment the amplification primers used are as follows:

### Forward 16S Primer:

5′-ATCGCCTACCGTGAC-barcode-AGAGTTTGATCMTGGCTCAG-3′ [SEQ ID NO: 1] and

### Reverse 16S Primer:

5′-ATCGCCTACCGTGAC-barcode-CGGTTACCTTGTTACGACTT-3′ [SEQ ID NO: 2]; or
16S-Mi341F forward primer 5′-CCTACGGGNGGCWGCAG-3′ [SEQ ID NO: 3] and 16S-Mi805R reverse primer 5′-GACTACHVGGGTATCTAATCC-3′ [SEQ ID NO: 4];
   optionally wherein when Nanopore technology is used, the primers are SEQ ID NO: 1 and 2; and where Illumina technology is used, the primers are SEQ ID NO: 3 and 4. Primers SEQ ID NO: 1 and 2 allow the amplification of the full length 16S (corresponding to 27F and 1492R positions). Primer SEQ ID NO: 3 and 4 generate an amplicon of around 460 bp.

In some embodiments the sequencing is performed using Illumina technology, optionally using a MiSeq device. In some embodiments the sequencing is performed using Nanopore technology. In some embodiments the sequencing is performed using both Illumina and Nanopore technology.

Identifying the species and/or genera in each of the control samples is within the skill of the skilled person. Particular methods are described in the examples, but essentially when identification involves sequencing, the sequences are compared to sequence databases and each microbe present identified at the species or genera level.

This gives information on the different species or genera of microbes, for example bacteria, that are present in the control samples. The sequencing information can also give an indication of the relative abundance of each of those microbes, for example bacteria. A higher number of reads that maps to a first species or genera versus a lower number of reads that maps to a second species or genera indicates that in the skin sample, the abundance of the first species was higher than the second species. In this way, the abundance of each species or genera can be quantified.

The microbial profile may show a difference in the number or type of species or genera that inhabit skin with a high level or a low level of the characteristic. The microbial profile may also show a difference in the relative abundance of the same species or genera between skin with a high level or a low level of the characteristic. The microbial profile may show that particular genera inhabit skin with a high level of the characteristic and a different set of genera inhabit skin with a low level of the characteristic. In some embodiments, it is the relative difference between the abundance or percentage abundance of genera that inhabit skin with a high level of the characteristic and the abundance or percentage abundance of genera that inhabit skin with a low level of the characteristic.

In some instances the microbial profile may comprise simply the panel of different microbes present in the two sets of control samples, for example different bacterial genera or species that are found in a skin with a high level of the characteristic versus the panel of different microbes, for example different bacterial genera or species that are found in a skin with a low level of the characteristic. For example a simple profile may be that subjects with a high level of the characteristic tend to have bacteria genera A, B, C and D on their skin, whereas subjects with a low level of the characteristic tend to have instead bacterial genera E, F, G and H on their skin.

Accordingly, in some embodiments the microbial profile is a profile of the different species or genera, for example different bacterial genera or species that are found on skin with a high level of the characteristic and versus a low level of the characteristic.

The two control microbial profiles—a first control microbial profile generated from samples taken from a number of subjects with a high level of the characteristic, and a second control microbial profile generated from a samples taken from a number of subjects with a low level of the characteristic—can be compared to one another by any known means. In some instances, it may be possible to identify a predictive microbial signature simply by looking at the two profiles. In other instances, the use of computer technology, for example an algorithm, may be used to identify the predictive signature. An example of such an approach is given in the Examples.

However, a more appropriate microbial signature takes into account the relative abundance of each of the species or genera, particularly since it is possible that in the example above a subject with a high level of the characteristic may also have some species or genera F-H, and subjects with a low level of the characteristic may have some species or genera A-D on their skin. Taking account of the percentage abundance of each of these genera or species gives an indication of whether overall a subject has a skin with a greater abundance of the genera or species that are associated with a high level of the characteristic, or if overall the subject has a skin with a greater abundance of the genera or species that are associated with a low level of the characteristic.

Accordingly, in one embodiment, once the identity of the microbes, for example bacteria, in the control samples have been ascertained (at the genera or species level), the percentage abundance of each species or genera in each sample is determined. FIGS. 6 and 7 show examples of the percentage abundance of genera obtained from the skin of two subjects. In some preferred embodiments the mean of the percentage abundance for each species or genera in the two populations (high or low level of the characteristic) is generated. This provides an average abundance of each of the relevant genera in the group of subjects with a high level of the characteristic, and an average abundance of the relevant genera or species of subjects with a low level of the characteristic. For example, such data could be as follows:

| | High level of characteristic | Low level of characteristic |
|---|---|---|
| | (mean % abundance of n | (mean % abundance of n |
| Genera | samples) | samples) |
| A | 20 | 2 |
| B | 10 | 3 |
| C | 30 | 5 |
| D | 20 | 10 |
| E | 2 | 20 |
| F | 3 | 10 |
| G | 5 | 30 |
| H | 10 | 20 |

Accordingly, in one embodiment, the method comprises:
c) generating a mean percentage abundance of each genera from the control samples showing a high level of the characteristic; and
d) generating a mean percentage abundance of each genera from the control samples showing a low level of the characteristic.

From these mean values, it is possible to identify the species or genera of microbes, for example genera of bacteria, that are more prevalent in skin with a high level of the characteristic, and those that are more prevalent in skin with a low level of the characteristic. In the example above it is possible to see that genera A-D are more prevalent in the high level condition, and genera E-H are more prevalent in the low level condition. However, such patterns, or signatures, are not necessarily easy to spot and the use of algorithms can be used to identify the patterns that are statistically significant. The examples provide a description of one way of doing this that uses the DESeq2 (Bioconductor) package. This compares the relative abundances of each of the species/genera and identifies which species or genera are significantly associated with each characteristic (high or low level). Other suitable packages are available.

It is advantageous if the species or genera that are part of the final signature do not include those that are only found very rarely in the skin samples tested. Accordingly, in one embodiment, species or genera are discarded from further analysis if they were not identified in more than 10%, 15%, 20%, 25%, 30%, 35%, 40%, 45%, 50%, 55%, 60%, 65%, 70%, 75% or 80% of control samples, for example:
species or genera are discarded from further analysis if they were not detected in more than 10%, 15%, 20%, 25%, 30%, 35%, 40%, 45%, 50%, 55%, 60%, 65%, 70%, 75% or 80% of control samples taken from subjects with a high level of the characteristic; and/or
species or genera are discarded from further analysis if they were not detected in more than 10%, 15%, 20%, 25%, 30%, 35%, 40%, 45%, 50%, 55%, 60%, 65%, 70%, 75% or 80% of control samples taken from subjects with a low level of the characteristic.

In one particular embodiment shown in the examples, microorganisms with a pvalue<0.05, a relative abundance higher than 0.1% and that were found in at least 30% of the samples were then classified using a machine learning approach based on a RandomForest classifier (RamdomForest package from R) to define more precisely which bacteria is the more relevant to characterize a specific skin condition. The Random Forest approach uses thousands of phylogenetic trees to define which bacterial genera allow the better to discriminate samples according to the main variable (i.e. the level of the characteristic).

It is also advantageous if the genera or species are detectable by a range of different methods. It is known that some sequencing methods are able to generate long sequence reads, and some are only able to generate shorter reads. The skilled person is aware of this and can design amplification and sequencing primers accordingly to ensure that the appropriate unique sequences are sequenced. Following the initial 16S rRNA sequencing to identify the microbes such as bacteria that are present, it is then possible to design specific primers that can target just those key genera or species that make up the microbial signature and which would be used for the actual testing of subject samples. i.e. although when initially establishing the microbial signature non-specific primers will generally be used, i.e. primers that can amplify or sequence the 16S rRNA sequence of all microbes for example bacteria, when actually using the signature to provide a recommendation as to which skin care products are most suitable, it is not necessary to use these primers, and primers specific to the relevant microbes can be used. These primers can also be designed to work with any type of sequencing methods, for example Illumina methods or Nanopore methods. The invention provides such primers and such combinations of primers to amplify and sequence any of the combinations of the microbes described herein.

Once the microbes e.g. bacteria that are more prevalent in skin with a high level of the condition or a those that are more prevalent in skin with a low level of the condition have been identified, in some embodiments it is advantageous to remove from further analysis any microbes e.g. bacteria that cannot be readily amplified and/or sequenced using most of the commonly used sequencing methods. Accordingly, in some embodiments, once the microbes e.g. bacteria that are more prevalent in skin with a high level of the condition or a those that are more prevalent in skin with a low level of the condition have been identified, any microbes for example bacteria for example genera or species of bacteria that cannot be sequencing using Illumina and/or Nanopore technology are removed from further analysis. The aim of this is to make sure that the microbial signature can be used with as many different sequencing systems as possible.

Although differences may exist between which species/genera are capable of being amplified and sequenced with Illumia versus Nanopore, it is considered that these differences are largely due to the primers used not being optimised for both conditions simultaneously. It is considered that all species and genera of microbes e.g. bacteria can be sequenced by both Illumina and Nanopore, provided that appropriate primers are used. The skilled person is well versed in the design of primers for a range of situations and is capable of designing appropriate primers. The Examples detail means of putting the invention into practice using both Illumina and Nanopore technology.

Once the mean percentage abundance of each genera/species has been calculated, and those genera or species that are more prevalent in skin with a high or low level of the characteristic (step (e) and (f) above) have been identified, these mean percentages can be used directly as the microbial signature and test samples can be compared to each of these values.

However, a further embodiment that produces a microbial signature that when used to predict the level of a characteristic of the skin of a test subject involves comparing a single test value to the signature, and so can be considered to be simpler for the skin care technician to use, comprises:
g)
i) for the control skin samples showing a high level of the characteristic, summing the mean percentage abundance of each genera identified in (e) and summing the mean percentage abundance of each genera identified in (f); and
ii) calculating the ratio of the sum of the mean percentage abundance of each genera identified in (e) to the sum of the mean percentage abundance of each genera identified in
(f) giving an upper threshold ratio; and
   iii) for the control skin samples showing a low level of the characteristic, summing the mean percentage abundance of each genera identified in (e) and summing the mean percentage abundance of each genera identified in (f); and
   iv) calculating the ratio of the sum of the mean percentage abundance of each genera identified in (e) to the sum of the mean percentage abundance of each genera identified in (f) giving a lower threshold

Using the exemplary data in the table above, this would result in:
High level of the characteristic samples: Sum of mean percentage abundances associated with high level of characteristic=20+10+30+20=80. Sum of percentage abundances associated with low level of characteristic=2+3+5+10=20
Upper threshold ratio=80/20=4
Low level of the characteristic samples: Sum of mean percentage abundances associated with high level of characteristic=2+3+5+10=20. Sum of percentage abundances associated with low level of characteristic=20+10+30+20=80.
Lower threshold ratio=20/80=0.25

The threshold ratios define the upper and lower ratios above which a test sample is considered to have a high level of the characteristic, below which a test sample is considered to have a low level of the characteristic, and where a test sample as a ratio that is in between the two thresholds, the sample is considered to have an intermediate level of the characteristic. In this case, a test subject having a test ratio of 4 or above would be considered to have skin with a high level of the characteristic; a ratio of 0.25 or less would be considered to have a skin with a low level of the characteristic; and a ratio of above 0.25 but less than 4 would be an intermediate level of the characteristic.

The terms “high” and “low” are standard in the field. For example, the skilled person would understand what is considered to be a “high” level of sebum or a “low” level of sebum.

Accordingly, in one embodiment, a microbial signature determined by the method is a set of microbial species or genera, for example bacterial genera, that are considered indicative of a high level of the characteristic, and a set of microbial species or genera, for example bacterial genera, that are considered indicative of a low level of the characteristic, and two threshold ratios as defined above. To use this signature, a test sample will be processed to determine the percentage abundance of each species or genera in the signature, and the ratio of abundance of species or genera indicative of a high level of the condition to the abundance of species or genera indicative of a low level of the condition is calculated and compared to the two threshold ratios to predict the level of the characteristic in the skin of the subject-high, low or intermediate.

It will be clear to the skilled person that the microbial signature can be used in ways that do not involved the generation of the threshold ratios described above. For example, the initial microbial profile generated from samples from skin with a high level of the condition can be compared to the microbial profiles obtained from samples taken from skin with a low level of the condition by any means. For example, it may be possible to identify unique subsets of microorganisms that are only ever associated with a high or a low level of the condition. In other embodiments the microbial signature may simply be the sum of the abundances of each genera associated with a high level of the condition i.e. it may not be necessary to compare it to the abundance of any species or genera such as genera of bacteria that are associated with the low level of the condition. In such instances it may not be necessary to generate threshold ratios, since when used in practice the skilled person testing a subjects skin may simply only need to determine the presence of one or more species or genera, for example bacterial genera.

Accordingly, in one embodiment a test sample comprising genera of microbes that are more prevalent in skin showing a high level of the characteristic is considered to predict that the skin has a high level of the characteristic, and wherein a test sample comprising genera of microbes that are more prevalent in skin showing a low level of the characteristic is considered to predict that the skin has a low level of the characteristic. In some embodiments the presence of microbes that are more prevalent in skin showing a high level of the characteristic is considered to predict that the skin has a high level of the characteristic, and in the same or other embodiments the presence of genera of microbes that are more prevalent in skin showing a low level of the characteristic is considered to predict that the skin has a low level of the condition. In the same or other embodiments, the sum of the abundance of species or genera that are more prevalent in skin with a high level of the condition is indicative of skin having a high level of the condition, for example if the sum of the abundance is above some threshold value associated with a high level of the condition. Alternatively or additionally, the sum of the abundance of species or genera that are more prevalent in skin with a low level of the condition is indicative of skin having a low level of the condition, for example if the sum of the abundance is above some threshold value associated with a low level of the condition.

As described above, there can be differences between the species/genera that are detectable using Illumina versus Nanopore. In one embodiment the microbial signature generated is tested on a range of samples, using both Illumina and Nanopore technology. In some embodiments the microbial signature generated provides the same prediction (i.e. high, low or intermediate level of the characteristic) in at least than 60%, 65%, 70%, 75%, 80%, 85%, 90%, 92%, 94%, 96%, 98% or 100% of test samples when the sample is processed using Illumina and Nanopore technology.

In some embodiments a correlation between a prediction obtained using Illumina and a prediction obtained using Nanopore is considered to be a true correlation when a LOW Illumina prediction corresponds to a LOW Nanopore prediction and when a HIGH Illumina prediction corresponds to a HIGH Nanopore prediction. In some embodiments, an intermediate prediction obtained using one of the technologies can be considered to be a true correlation to the other technology if it corresponds to an intermediate, HIGH or LOW prediction in the other technology.

The methods of the invention can be used to generate a microbial signature that can predict any skin characteristic, for example any cosmetic skin characteristic or a medical skin characteristic.

In some embodiments the cosmetic skin characteristics comprise skin sensitivity, sebum level, hydration, brown spots, aged skin, and level of barrier function. Accordingly, in one embodiment, the method comprises a combination of microbial signatures that are predictive of at least two of the following:
The high level of a characteristic is a high level of sensitivity and the low level of a characteristic is a low level of sensitivity;
The high level of a characteristic is a high level of sebum and the low level of a characteristic is a low level of sebum;
The high level of a characteristic is a high level of hydration and the low level of a characteristic is a low level of hydration;
The high level of a characteristic is a high level of brown spots and the low level of a characteristic is a low level of brown spots;
The high level of a characteristic is a high level of aged skin and the low level of a characteristic is a low level of aged skin; and/or
The high level of a characteristic is a high level of barrier function and the low level of a characteristic is a low level of barrier function.

In some embodiments the microbial signature (or combination of microbial signatures) is predictive of all of:
The high level of a characteristic is a high level of sensitivity and the low level of a characteristic is a low level of sensitivity;
The high level of a characteristic is a high level of sebum and the low level of a characteristic is a low level of sebum;
The high level of a characteristic is a high level of hydration and the low level of a characteristic is a low level of hydration;
The high level of a characteristic is a high level of brown spots and the low level of a characteristic is a low level of brown spots;
The high level of a characteristic is a high level of aged skin and the low level of a characteristic is a low level of aged skin; and
The high level of a characteristic is a high level of barrier function and the low level of a characteristic is a low level of barrier function.

All of these characteristics are routinely assessed in the cosmetic industry and the skilled person is well aware of methods to determine the level of each of these parameters.

For example the sebum level can be measured using a Sebumeter®. It is a photometric method. A synthetic ribbon, which becomes transparent when in contact with absorbed lipids, is applied to the measurement zone for precisely 30 seconds. Its transparency increases proportionally with the quantity of sebum from the hydrolipidic film with which it is in contact. A reflectometry recording is used to quantify the increase of the light transmitted and to determine the total mass of the lipids excreted by the surface unit (in μg/cm²). For example, using said Sebumenter, low sebum can be consider=>23 and high sebum levels can be consider=>63.

The hydration level may be measured using a Corneometer®. The stratum corneum hydration causes changing in its electrical characteristics. The stratum corneum is like a dielectric corps. Any modifications of its hydration statement cause a variation of the electric capacity measured by a condenser. Higher is the hydration, higher is the electric capacity because its dipolar nature increases the electric permittivity of the environment and its conductibility. Measurement was performed by the Corneometer® CM 825 TM (Courage & Khazaka electronics). The probe linked to a condenser allows applying at all the time the same pressure on the tegument in order to not disturb the measures and obtain good experimental conditions reproducibility. For example, using said Corneometer, low hydration can be considered <=45.4 and high hydratation=>50.

The Trans-Epidermal Water Loss (TEWL) gives an indication of barrier function, so the level of barrier function can be measured using a Tewameter®. TEWL is the passive diffusion of water through the stratum corneum from the inside of the body to the outside. Its measurement makes it possible to appreciate the integrity of the barrier function. A probe delimiting a cylindrical chamber in contact with the skin is used to measure the gradient of water vapor established on the cutaneous surface, an open chamber in order to partially overcome variations in environmental conditions. The unit of measurement is g/m²/h. A high TEWL (high as used in the data in the examples) indicates a low level of barrier function as used in the text; and a low TEWL as used in the data in the examples and figures indicates a high level of barrier function as used in the text. For example, TEWL (barrier function) can be considered low <=11.2; and high=>16.2.

The sensitive skin phenotype can be established on the basis of the adverse sensory response to capsaicin test.

Brown spots (pigmentation and discoloration on and beneath the surface of the skin) can be measured using a Visia CR 2.3® from Canfield® imaging systems. The VISIA allows taking pictures with different types of illuminations and a very rapid capture of images. A series of photos taken under multi-spectral imaging and analysis allow capturing visual information affecting appearance of the skin. Canfield's RBX® Technology separates the unique color signatures of Red and Brown skin components for unequaled visualization of conditions that result in color concentration, such as spider veins, hyperpigmentation, inflammation and other conditions. Because of the difference of the size of faces of the volunteers, a normalization of the number of spots has been made is using the surface considered for the measurement. For example, low spot area can be considered=>1061; and high spot area=>2237.

By “level of aged skin” we mean the age of the skin. This language is used to be consistent with the rest of the terminology used. However, it will be clear to the skilled person that, for example in the method of identifying a microbial signature that is predictive of a high or low level of aged skin, we mean that the signature can predict the apparent age of the skin. Once the signature is determined (i.e. which species/genera are associated with older versus younger skin), it will be possible to predict the age of the skin of a test sample from a subject. This can then be compared to the actual age of the test subject and it can be established if the subject has prematurely aged skin, or not. For example low age can be considered <=25; and high age=>42.

The method can be used to identify a microbial signature that can predict the level of just one of skin sensitivity, sebum level, hydration, brown spots, aged skin, and level of barrier function. However, the method can also be used to generate a combination of microbial signatures that can predict the level of more than one of, or all of, skin sensitivity, sebum level, hydration, brown spots, aged skin, and level of barrier function.

Accordingly in one embodiment the method is a method of identifying a microbial signature predictive of either a high level or low level of a least a 2, 3, 4, 5, 6, 7, 8, 9, 10 or more characteristics, optionally wherein the characteristics are selected from skin sensitivity, sebum level, hydration, brown spots, aged skin, and level of barrier function.

It is clearly advantageous if the microbial signature that predicts a high or low level of a first characteristic is different to the microbial signature that predicts a high or low level of a second, third, fourth, fifth or sixth characteristic. Accordingly in one embodiment the method comprises identifying a first microbial profile predictive of a high level of a first skin characteristic or a low level of a first skin characteristic, and a second microbial profile predictive of a high level of a second skin characteristic or a low level of a second characteristic, the first and the second microbial signatures are different (and optionally a third, fourth, fifth, sixth, seventh, eighth, ninth, tenth or more characteristic).

As described above, the microbial sample can be a sample of any one or more types of microorganisms, for example may be a sample of any one or more of bacteria, yeast and fungi in the sample.

As described above, the microbial profile can be a profile of any one or more types of microorganisms, for example may be a profile of any one or more of bacteria, yeast and fungi in the sample. In preferred embodiments the microbial profile is a bacterial profile.

In some embodiments the microbial profile is a microbial profile of the individual species within the sample. In other embodiments the microbial profile is a profile of the genera of the relevant organisms in the sample. For example, in some embodiments, the microbial profile is a profile of the genera of bacteria in the sample.

In addition to providing a method of establishing or identifying a microbial signature that predicts the level (high or low) or a skin characteristic, the invention also provides:
a method of predicting whether a subject has a high level of a first skin characteristic, a low level of the first skin characteristic, or an intermediate level of the first skin characteristic in a human subject wherein the method comprises:
a) providing a skin microbial sample taken from a subject and generating a test microbial profile from the skin microbial sample;
b) comparing the test microbial profile with a microbial signature that is predictive of a high level of the first characteristic or a low level of the first characteristic; and
c) determining whether the test microbial profile is that of skin with a high level of the first characteristic, a low level of the first characteristic, or an intermediate level of the first characteristic.

Preferences for this aspect of the invention are as described elsewhere, for example preferences for the characteristics, samples etc.

In some embodiments the microbial signature is established or identified using the methods of the invention described herein.

In some embodiments, generating the test microbial profile comprises:
a ii) detecting the presence of and/or determining the abundance of and/or calculating the percentage abundance of each genera of a microbial signature that is predictive of a high level of the first skin characteristic or a low level of the first skin characteristic; and optionally
a iii) summing the percentage abundance of each genera that is predictive of a high level of the first characteristic; and summing the percentage abundance of each genera that is predictive of a low level of the first characteristic.

In some embodiments the method further comprises:
a iv) calculating the ratio of the sum of the percentage abundance of each genera that is predictive of the high level of the first characteristic to the sum of the percentage abundance of each genera that is predictive of a low level of the first characteristic to generate a test ratio. In some embodiments the method also comprises
a v) comparing the test ratio of (a iv) to a pre-defined upper threshold ratio and a pre-defined lower threshold ratio, wherein a test ratio that is greater than the upper threshold ratio predicts that the subject has skin with a high level of the first characteristic; a test ratio that is lower than the lower threshold ratio predicts that the subject has skin with a low level of the first characteristic; and a test ratio between the upper and lower threshold ratios predicts an intermediate skin characteristic.

In some embodiments, instead of or as well as steps (a iv) and (a v), the method can comprise comparing the presence, abundance or percentage abundance of the genera predictive of a high level of a low level of the characteristic determined in (a ii) or sum of the abundance or percentage abundance of (a iii) to a microbial signature.

Typically, by “a genera that is predictive of” we include the meaning that the higher the abundance of said genera, the stronger is the prediction of that characteristic. So a high abundance of a genera that is predictive of a high level of the characteristic is more predictive of the high level of the characteristic than a lower abundance of the genera.

It will be apparent to the skilled person that where a high abundance of a particular genera or species, for example genera of bacteria is indicative of a high level of a characteristic, a low abundance of the same particular genera or species, for example genera of bacteria is indicative of a low level of the characteristic.

In some embodiments, the genera or species that predict a high level of a characteristic and the genera or species that predict a low level of a characteristic are the same species or genera, and the abundance is what determines if a high level or a low level is predicted. In preferred embodiments, the genera or species that predict a high level of a characteristic and the genera or species that predict a low level of a characteristic are different species or genera. The abundance of the species or genera that predicts a particular level of a characteristic may be high or low.

In a preferred embodiment, the genera or species that predict a high level of a characteristic and the genera or species that predict a low level of a characteristic are, and in each case a high abundance of the species or genera predicts the high level or the low level of the condition. For example a first high abundance of a first set of species or genera predicts a high level of the characteristic, and a second high abundance of a second set of species or genera predicts a low level of the characteristic. In some embodiments, the first abundance and second abundance are compared to determine the characteristic of the skin. In this way it is possible to then determine which set of species or genera has the overall highest abundance, and so which level, high or low, of the characteristic is predicted. The comparison may be in the form of a ratio, or a subtraction.

In some embodiments, the upper and lower threshold ratios are determined according to the methods of the invention described herein.

In some embodiments, the microbial signature that is predictive of a high level of a first characteristic comprises any one of more of the following genera:
Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and Xanthomonas.

In the same or different embodiment, the microbial signature that is predictive of a low level of the first characteristic comprises any one of more of the following genera: Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and Xanthomonas.

In some embodiments:
The high level of a characteristic is a high level of sensitivity and the low level of a characteristic is a low level of sensitivity;
The high level of a characteristic is a high level of sebum and the low level of a characteristic is a low level of sebum;
The high level of a characteristic is a high level of hydration and the low level of a characteristic is a low level of hydration;
The high level of a characteristic is a high level of brown spots and the low level of a characteristic is a low level of brown spots;
The high level of a characteristic is a high level of aged skin and the low level of a characteristic is a low level of aged skin; and/or
The high level of a characteristic is a high level of barrier function and the low level of a characteristic is a low level of barrier function.

In particular embodiments, wherein the high level of the first characteristic is a high level of sebum and the low level of the first characteristic is a low level of sebum, and wherein the genera predictive of high sebum comprises any one or more or all of:
a) Cutibacterium and/or Eikenella;
b) Actinobacillus, Anaerococcus, Bacillus, Cutibacterium, Eikenella, Peptoniphilus, Snodgrassella, Staphylococcus and/or Turicella; and/or
c) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In the same or different embodiment, the high level of the first characteristic is a high level of sebum and the low level of the first characteristic is a low level of sebum, and the genera predictive of a low level of sebum comprises any one or more or all of:
a) Haemophilus, Kocuria and/or Neisseria;
b) Haemophilus, Kocuria, Neisseria and/or Paucibacter;
c) Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and/or Brochothix;
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In particular embodiments, the high level of the first characteristic is a high level of sebum and the low level of the first characteristic is a low level of sebum, and
a) the genera predictive of high sebum comprises any one or more or all of:
b) Cutibacterium and/or Eikenella
and the genera predictive of low sebum comprise any one or more or all of: Haemophilus, Kocuria, Neisseria.
c) the genera predictive of high sebum comprises any one or more or all of:
d) Cutibacterium and/or Eikenella
and the genera predictive of low sebum comprise any one or more or all of: Haemophilus, Kocuria, Neisseria, Paucibacter;
c) the genera predictive of high sebum comprises any one or more or all of:
Actinobacillus, Anaerococcus, Bacillus, Cutibacterium, Eikenella, Peptoniphilus, Snodgrassella, Staphylococcus and/or Turicella;
and the genera predictive of low sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus,
d) the genera predictive of a high level of sebum comprises any one or more or all of: Actinobacillus, Anaerococcus, Bacillus, Cutibacterium, Eikenella, Peptoniphilus, Snodgrassella, Staphylococcus and/or Turicella;
and the genera predictive of a low level of sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and/or Brochothix.

In some embodiments, the high level of a first characteristic is a high level of sensitivity and the low level of the first characteristic is a low level of sensitivity, and the genera predictive of a high level of sensitivity comprises any one or more or all of:
a) Corynebacterium and/or Kocuria;
b) Anaerococcus, Kocuria and/or Snodgrassella;
c) Actinobacillus, Anaerococcus, Corynebacterium, Filifactor, Kocuria, Roseomonas and/or Snodgrassella; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In the same or different embodiment, the high level of a first characteristic is a high level of sensitivity and the low level of the first characteristic is a low level of sensitivity, and the genera predictive of a low level of sensitivity comprises any one or more or all of:
a) Cutibacterium;
b) Cutibacterium and/or Staphylococcus;
c) Bacillus, Cutibacterium, Dolosigranulum, Eikenella, Staphylococcus and/or Stenotrophomonas;
d) Bacillus, Campylobacter, Cutibacterium, Delftia, Dolosigranulum, Eikenella, Frigoribacterium, Glutamicibacter, Moraxella, Serratia, Staphylococcus, Stenotrophomonas and/or Undibacterium;
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In particular embodiments, the high level of a first characteristic is a high level of sensitivity and the low level of the first characteristic is a low level of sensitivity, and
a) the genera predictive of a high level of sensitivity comprises any one or more or all of: Corynebacterium and/or Kocuria;
and the genera predictive of a low level of sensitivity comprise any one or more or all of: Cutibacterium.
b) the genera predictive of a high level of sensitivity comprises any one or more or all of: Anaerococcus, Kocuria and/or Snodgrassella.

And the genera predictive of a low level of sensitivity comprise any one or more or all of: Bacillus, Cutibacterium, Dolosigranulum, Eikenella, Staphylococcus and/or Stenotrophomonas
c) the genera predictive of a high level of sensitivity comprises any one or more or all of: Corynebacterium and/or Kocuria
and the genera predictive of a low level of sensitivity comprises any one or more or all of: Cutibacterium and/or Staphylococcus;
d) the genera predictive of a high level of sensitivity comprises any one or more or all of: Actinobacillus, Anaerococcus, Corynebacterium, Filifactor, Kocuria, Roseomonas and/or Snodgrassella;
and the genera predictive of a low level of sensitivity comprises any one or more or all of: Bacillus, Campylobacter, Cutibacterium, Delftia, Dolosigranulum, Eikenella, Frigoribacterium, Glutamicibacter, Moraxella, Serratia, Staphylococcus, Stenotrophomonas and/or Undibacterium.

In some embodiments, the high level of a first characteristic is a high level of hydration and the low level of the first characteristic is a low level of hydration, and the genera predictive of a high level of hydration comprises any one or more or all of:
a) Haemophilus and/or Snodgrassella;
b) Bacillus, Haemophilus and/or Kocuria;
c) Bacillus, Haemophilus, Kocuria, Paucibacter and/or Snodgrassella;
d) Allorhizobium, Amaricoccus, Bacillus, Brevundimonas, Falsirhodobacter, Haemophilus, Kocuria, Lautropia, Paucibacter and/or Snodgrassella; and/or
e) Acinetobacter, Actinobacillus, Aerococcus, Alloiococcus, Allorhizobium, Amaricoccus, Anaerococcus, Atopobium, Bacillus, Bergeyella, Brachybacterium, Campylobacter, Chryseobacterium, Brevibacterium, Brevundimonas, Brochothrix, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In the same or different embodiment, the high level of a first characteristic is a high level of hydration and the low level of the first characteristic is a low level of hydration, and the genera predictive of a low level of hydration comprises any one or more or all of:
a) Brochothrix and/or Pseudomonas;
b) Brochothrix, Cutibacterium, Pseudomonas and/or Staphylococcus;
c) Brochothrix, Cutibacterium, Pseudomonas, Serratia, Staphylococcus, Turicella and/or Undibacterium; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In particular embodiments, the high level of a first characteristic is a high level of hydration and the low level of the first characteristic is a low level of hydration, and
a) the genera predictive of a high level of hydration comprises any one or more or all of: Haemophilus and/or Snodgrassella.

And the genera predictive of a low level of hydration comprises any one or more or all of: Brochothrix and/or Pseudomonas;
b) the genera predictive of a high level of hydration comprises any one or more or all of: Bacillus, Haemophilus and/or Kocuria,
and the genera predictive of a low level of hydration comprises any one or more or all of: Brochothrix, Cutibacterium, Pseudomonas and/or Staphylococcus
c) the genera predictive of a high level of hydration comprises any one or more or all of: Bacillus, Haemophilus, Kocuria, Paucibacter and/or Snodgrassella
and the genera predictive of low hydration comprises any one or more or all of: Brochothrix, Cutibacterium, Pseudomonas and/or Staphylococcus
d) the genera predictive of a high level of hydration comprises any one or more or all of: Allorhizobium, Amaricoccus, Bacillus, Brevundimonas, Falsirhodobacter, Haemophilus, Kocuria, Lautropia, Paucibacter and/or Snodgrassella;
and the genera predictive of a low level of hydration comprises any one or more or all of: Brochothrix, Cutibacterium, Pseudomonas, Serratia, Staphylococcus, Turicella and/or Undibacterium.

In some embodiments the high level of a first characteristic is a high level of brown spots and the low level of the first characteristic is a low level of brown spots, and the genera predictive of high brown spots comprises any one or more or all of:
a) Eikenella;
b) Eikenella, Aerococcus, Xanthomonas and/or Brevibacterium;
c) Paucibacter, Turicella, Xanthomonas, Brevibacterium, Eikenella and/or Aerococcus;
d) Aerococcus, Brevibacterium, Eikenella, Klebsiella, Paucibacter, Turicella and/or Xanthomonas; and/or
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In the same or different embodiments, the high level of a first characteristic is a high level of brown spots and the low level of the first characteristic is a low level of brown spots, and the genera predictive of low brown spots comprises any one or more or all of:
a) Micrococcus and/or Paracoccus;
b) Kocuria, Micrococcus, Paracoccus and/or Bergeyella;
c) Alloiococcus, Bergeyella, Exiguobacterium, Kocuria, Micrococcus and/or Paracoccus; and/or
d) Acinetobacter, Actinobacillus, Aerococcus, Alloiococcus, Allorhizobium, Amaricoccus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In particular embodiments, the high level of a first characteristic is a high level of brown spots and the low level of the first characteristic is a low level of brown spots, and
a) the genera predictive of a high level of brown spots comprises any one or more or all of: Eikenella
and the genera predictive of a low level of brown spots comprises any one or more or all of: Micrococcus and/or Paracoccus;
b) the genera predictive of a high level of brown spots comprises any one or more or all of: Eikenella, Aerococcus, Xanthomonas, and/or Brevibacterium
and the genera predictive of a low level of brown spots comprises any one or more or all of: Kocuria, Micrococcus, Paracoccus and/or Bergeyella;
c) the genera predictive of a high level of brown spots comprises any one or more or all of: Paucibacter, Turicella, Xanthomonas, Brevibacterium, Eikenella, and/or Aerococcus
and the genera predictive of a low level of brown spots comprise any one or more or all of: Kocuria, Micrococcus, Paracoccus and/or Bergeyella
d) the genera predictive of a high level of brown spots comprises any one or more or all of: Aerococcus, Brevibacterium, Eikenella, Klebsiella, Paucibacter, Turicella and/or Xanthomonas
and the genera predictive of a low level of brown spots comprise any one or more or all of: Alloiococcus, Bergeyella, Exiguobacterium, Kocuria, Micrococcus and/or Paracoccus.

In some embodiments, the high level of a first characteristic is a high level of age and the low level of the first characteristic is a low level of age, and the genera predictive of a high level of age comprises any one or more or all of:
a) Eikenella and/or Brochothrix;
b) Eikenella, Aerococcus and/or Glutamicibacter;
c) Aerococcus, Atopobium, Bacillus, Brachybacterium, Brochothrix, Chryseobacterium, Eikenella, Eubacterium, Glutamicibacter and/or Pantoea; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In the same or different embodiment, the high level of a first characteristic is a high level of age and the low level of the first characteristic is a low level of age, and the genera predictive of a low level of age comprises any one or more or all of:
a) Finegoldia, Lawsonella and/or Peptoniphilus;
b) Finegoldia and/or Lawsonella;
c) Bergeyella, Cloacibacterium, Finegoldia, Lawsonella, Peptoniphilus and/or Undibacterium; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In particular embodiments, the high level of a first characteristic is a high level of age and the low level of the first characteristic is a low level of age, and
a) the genera predictive of a high level of age comprises any one or more or all of: Eikenella and/or Brochothrix
and the genera predictive of a low level of age comprises any one or more or all of: Finegoldia, Lawsonella and/or Peptoniphilus
b) the genera predictive of a high level of age comprises any one or more or all of: Eikenella, Aerococcus and/or Glutamicibacter
and the genera predictive of a low level of age comprises any one or more or all of: Finegoldia and/or Lawsonella
c) the genera predictive of a high level of age comprises any one or more or all of: Aerococcus, Atopobium, Bacillus, Brachybacterium, Brochothrix, Chryseobacterium, Eikenella, Eubacterium, Glutamicibacter and/or Pantoea;
and the genera predictive of a low level of age comprises any one or more or all of: Bergeyella, Cloacibacterium, Finegoldia, Lawsonella, Peptoniphilus and/or Undibacterium.

In some embodiments, the high level of a first characteristic is a high level of barrier function age and the low level of the first characteristic is a low level of barrier function, and the genera predictive of a low level of barrier function comprises any one or more or all of:
a) Cutibacterium;
b) Eikenella and/or Cutibacterium;
c) Brochothrix, Cutibacterium, Eikenella, Lactobacillus, Turicella and/or Veillonella; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;

In the same or different embodiment, the high level of a first characteristic is a high level of barrier function age and the low level of the first characteristic is a low level of barrier function, and the genera predictive of a high level of barrier function comprises any one or more or all of:
a) Paracoccus and/or Brevundimonas;
b) Micrococcus, Paracoccus, Brevundimonas, Bacillus and/or Xanthomonas;
c) Enhydrobacter, Micrococcus, Brevundimonas, Paracoccus and/or Bacillus;
d) Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and/or Xanthomonas; and/or
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

In particular embodiments, the high level of a first characteristic is a high level of barrier function age and the low level of the first characteristic is a low level of barrier function, and
a) the genera predictive of a low level of barrier function comprises any one or more or all of: Cutibacterium
and the genera predictive of a high level of barrier function comprises any one or more or all of: Paracoccus and/or Brevundimonas
b) the genera predictive of a low level of barrier function comprises any one or more or all of: Eikenella and/or Cutibacterium
and the genera predictive of a high level of barrier function comprises any one or more or all of: Micrococcus, Paracoccus, Brevundimonas, Bacillus and/or Xanthomonas
c) the genera predictive of a low level of barrier function comprises any one or more or all of: Eikenella and/or Cutibacterium
and the genera predictive of a high level of barrier function comprises any one or more or all of: Enhydrobacter, Micrococcus, Brevundimonas, Paracoccus and/or Bacillus
d) the genera predictive of a low level of barrier function comprises any one or more or all of: Brochothrix, Cutibacterium, Eikenella, Lactobacillus, Turicella and/or Veillonella
and the genera predictive of a high level of barrier function comprises any one or more or all of: Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and/or Xanthomonas.

In particular embodiments, the high level of a first characteristic is a high level of aged skin linked to smoking and the low level of the first characteristic is a low level of aged skin linked to smoking, and wherein the genera predictive of a high level of aged skin linked to smoking comprises:
a) Micrococcus, Bacillus, Eikenella, Gemella, Corynebacterium, Finegoldia, Cutibacterium and/or Anaerococcus;
In particular embodiments, the microbiota is measured in the check, the forehead and/or the scalp.

Preferences for obtaining a microbial profile are as described herein and above and include for example sequencing a region of the microbial genome. In some embodiments, as described elsewhere herein, said region of the bacterial genome is a region of the 16S rRNA variable region, optionally the V3V4 variable region. In some embodiments the primers used for amplification and/or sequences are:

Forward 16S primer:
[SEQ ID NO: 1]
5′-ATCGCCTACCGTGAC-barcode-
AGAGTTTGATCMTGGCTCAG-3′
and
Reverse 16S primer:
[SEQ ID NO: 2]
5′-ATCGCCTACCGTGAC-barcode-
CGGTTACCTTGTTACGACTT-3′;
or
16S-Mi341F forward primer
[SEQ ID NO: 3]
5′-CCTACGGGNGGCWGCAG-3′
and
16S-Mi805R reverse primer
[SEQ ID NO: 4]
5′-GACTACHVGGGTATCTAATCC-3′;
(N means “any bases” (A, T, G or C),
W is for weak = A or T, M for amino = A or C)

optionally wherein when Nanopore technology is used, the primers are SEQ ID NO: 1 and 2; and where Illumina technology is used, the primers are SEQ ID NO: 3 and 4. Primers SEQ ID NO: 1 and 2 allow the amplification of the full length 16S (corresponding to 27F and 1492R positions). Primer SEQ ID NO: 3 and 4 generate an amplicon of around 460 bp.

In some instances the sequencing is performed by Illumina sequencing, optionally using a MiSeq device. In other instances the sequencing is performed using nanopore sequencing technology.

It is also possible to generate a test microbial profile by culturing the microbes from the sample. In one embodiment the method of obtaining the test microbial profile is the same as the method used to obtain the control microbial profiles that were used to generate the microbial signature.

Preferences for the microbes are as described elsewhere herein. In some embodiments the microbial sample comprises any one or more of bacteria, yeast and fungi. Preferably the microbial sample comprises bacteria.

The microbial profile may be a profile of any one or more of bacteria, yeast and/or fungi, preferably the microbial profile is a bacterial profile.

It will be clear from the disclosure herein that the invention also provides a method for categorising skin type into each of the following characteristics:
a) a low level of sensitivity, a high level of sensitivity, or an intermediate level of sensitivity;
b) a low level of sebum, a high level of sebum, or an intermediate level of sebum;
c) a low level of hydration, a high level of hydration, or an intermediate level of hydration;
d) a low level of brown spots, a high level of brown spots, or an intermediate level of brown spots;
e) a low level of aged skin, a high level of aged skin, or an intermediate level of aged skin;
f) a low level of barrier function, a high level of barrier function, or an intermediate level of barrier function.

In some embodiments the method comprises determining the percentage abundance, abundance, or presence of bacteria from each of the following genera in a skin microbial sample:
Brevundimonas, Brochothrix, Corynebacterium, Cutibacterium, Eikenella, Finegoldia, Haemophilus, Kocuria, Lawsonella, Micrococcus, Neisseria, Paracoccus, Peptoniphilus, Pseudomonas and Snodgrassella.

The invention also provides a method of categorising skin type into a low level of sensitivity, a high level of sensitivity, or an intermediate level of sensitivity, wherein the method comprises determining the percentage abundance, abundance, or presence of bacteria from each of the following genera in a skin microbial sample:
a) Corynebacterium, Kocuria and Cutibacterium; optionally wherein
   the genera predictive of a high level of sensitivity comprises any one or more or all of Corynebacterium and/or Kocuria; and
   the genera predictive of a low level of sensitivity comprise Cutibacterium;
b) Anaerococcus, Kocuria, Snodgrassella, Bacillus, Cutibacterium, Dolosigranulum, Eikenella, Staphylococcus and Stenotrophomonas; optionally wherein
   the genera predictive of a high level of sensitivity comprises any one or more or all Anaerococcus, Kocuria and/or Snodgrassella; and of:
   the genera predictive of a low level of sensitivity comprise any one or more or all of: Bacillus, Cutibacterium, Dolosigranulum, Eikenella, Staphylococcus and/or Stenotrophomonas;
c) Corynebacterium, Kocuria, Cutibacterium and Staphylococcus; optionally wherein
   the genera predictive of a high level of sensitivity comprises any one or more or all of: Corynebacterium and/or Kocuria; and
   the genera predictive of a low level of sensitivity comprises any one or more or all of: Cutibacterium and/or Staphylococcus;
d) Actinobacillus, Anaerococcus, Corynebacterium, Filifactor, Kocuria, Roseomonas Snodgrassella, Bacillus, Campylobacter, Cutibacterium, Delftia, Dolosigranulum, Eikenella, Frigoribacterium, Glutamicibacter, Moraxella, Serratia, Staphylococcus, Stenotrophomonas and Undibacterium; optionally wherein
   the genera predictive of a high level of sensitivity comprises any one or more or all of: Actinobacillus, Anaerococcus, Corynebacterium, Filifactor, Kocuria, Roseomonas and/or Snodgrassella; and
   the genera predictive of a low level of sensitivity comprises any one or more or all of: Bacillus, Campylobacter, Cutibacterium, Delftia, Dolosigranulum, Eikenella, Frigoribacterium, Glutamicibacter, Moraxella, Serratia, Staphylococcus, Stenotrophomonas and Undibacterium;

The invention also provides a method of categorising skin type into a low level of sebum, a high level of sebum, or an intermediate level of sebum, wherein the method comprises determining the percentage abundance, abundance, or presence of bacteria from each of the following genera in a skin microbial sample:
a) Cutibacterium, Eikenella Haemophilus, Kocuria and Neisseria; optionally wherein
   the genera predictive of high sebum comprises any one or more or all of: Cutibacterium and/or Eikenella; and
   the genera predictive of low sebum comprise any one or more or all of: Haemophilus, Kocuria, Neisseria;
b) Cutibacterium, Eikenella, Haemophilus, Kocuria, Neisseria and Paucibacter; optionally wherein
   the genera predictive of high sebum comprises any one or more or all of: Cutibacterium and/or Eikenella; and
   the genera predictive of low sebum comprise any one or more or all of: Haemophilus, Kocuria, Neisseria, Paucibacter;
c) Actinobacillus, Anaerococcus, Bacillus, Cutibacterium, Eikenella, Peptoniphilus, Snodgrassella, Staphylococcus Turicella, Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, and Streptococcus; optionally wherein
   the genera predictive of high sebum comprises any one or more or all of: Actinobacillus, Anaerococcus, Bacillus, Cutibacterium, Eikenella, Peptoniphilus, Snodgrassella, Staphylococcus and/or Turicella; and
   the genera predictive of low sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus;
d) Actinobacillus, Anaerococcus, Bacillus, Cutibacterium, Eikenella, Peptoniphilus, Snodgrassella, Staphylococcus, Turicella Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and Brochothix; optionally wherein
   the genera predictive of a high level of sebum comprises any one or more or all of: Actinobacillus, Anaerococcus, Bacillus, Cutibacterium, Eikenella, Peptoniphilus, Snodgrassella, Staphylococcus and/or Turicella;
and
   the genera predictive of a low level of sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, a, Neisseria, Paracoccus, Paucibacter, Photobacterium, Porphyromonas, Roseomonas, Streptococcus and/or Brochothix.

The invention provides a method of categorising skin type into a low level of hydration, a high level of hydration, or an intermediate level of hydration, wherein the method comprises determining the percentage abundance, abundance, or presence of bacteria from each of the following genera in a skin microbial sample:
a) Haemophilus, Snodgrassella, Brochothrix and Pseudomonas; optionally wherein
   the genera predictive of a high level of hydration comprises any one or more or all of: Haemophilus and/or Snodgrassella; and
   the genera predictive of a low level of hydration comprises any one or more or all of: Brochothrix and/or Pseudomonas;
b) Bacillus, Haemophilus, Kocuria, Brochothrix, Cutibacterium, Pseudomonas and Staphylococcus; optionally wherein
   the genera predictive of a high level of hydration comprises any one or more or all of: Bacillus, Haemophilus and/or Kocuria; and
   the genera predictive of a low level of hydration comprises any one or more or all of: Brochothrix, Cutibacterium, Pseudomonas and/or Staphylococcus;
c) Bacillus, Haemophilus, Kocuria, Paucibacter and/or Snodgrassella; optionally wherein
   the genera predictive of a high level of hydration comprises any one or more or all of: Bacillus, Haemophilus, Kocuria, Paucibacter, Snodgrassella, Brochothrix, Cutibacterium, Pseudomonas and Staphylococcus; and
   the genera predictive of low hydration comprises any one or more or all of: Brochothrix, Cutibacterium, Pseudomonas and/or Staphylococcus;
e) Allorhizobium, Amaricoccus, Bacillus, Brevundimonas, Falsirhodobacter, Haemophilus, Kocuria, Lautropia, Paucibacter, Snodgrassella; Brochothrix, Cutibacterium, Pseudomonas, Serratia, Staphylococcus, Turicella and Undibacterium; optionally wherein
   the genera predictive of a high level of hydration comprises any one or more or all of: Allorhizobium, Amaricoccus, Bacillus, Brevundimonas, Falsirhodobacter, Haemophilus, Kocuria, Lautropia, Paucibacter and/or Snodgrassella; and
   the genera predictive of a low level of hydration comprises any one or more or all of: Brochothrix, Cutibacterium, Pseudomonas, Serratia, Staphylococcus, Turicella and/or Undibacterium.

The invention provides a method of categorising skin type into a low level of brown spots, a high level of brown spots, or an intermediate level of brown spots, wherein the method comprises determining the percentage abundance, abundance, or presence of bacteria from each of the following genera in a skin microbial sample:
a) Eikenella, Micrococcus and Paracoccus; optionally wherein
   the genera predictive of a high level of brown spots comprises any one or more or all of: Eikenella; and
   the genera predictive of a low level of brown spots comprises any one or more or all of: Micrococcus and/or Paracoccus;
b) Eikenella, Aerococcus, Xanthomonas, Brevibacterium, Kocuria, Micrococcus, Paracoccus and Bergeyella; optionally wherein
   the genera predictive of a high level of brown spots comprises any one or more or all of: Eikenella, Aerococcus, Xanthomonas, and/or Brevibacterium; and
   the genera predictive of a low level of brown spots comprises any one or more or all of: Kocuria, Micrococcus, Paracoccus and/or Bergeyella;
c) Paucibacter, Turicella, Xanthomonas, Brevibacterium, Eikenella, Aerococcus, Kocuria, Micrococcus, Paracoccus and Bergeyella; optionally wherein
   the genera predictive of a high level of brown spots comprises any one or more or all of: Paucibacter, Turicella, Xanthomonas, Brevibacterium, Eikenella, and/or Aerococcus; and
   the genera predictive of a low level of brown spots comprise any one or more or all of: Kocuria, Micrococcus, Paracoccus and/or Bergeyella;
d) Aerococcus, Brevibacterium, Eikenella, Klebsiella, Paucibacter, Turicella Xanthomonas, Alloiococcus, Bergeyella, Exiguobacterium, Kocuria, Micrococcus and Paracoccus; optionally wherein
   the genera predictive of a high level of brown spots comprises any one or more or all of: Aerococcus, Brevibacterium, Eikenella, Klebsiella, Paucibacter, Turicella and/or Xanthomonas; and
   the genera predictive of a low level of brown spots comprise any one or more or all of: Alloiococcus, Bergeyella, Exiguobacterium, Kocuria, Micrococcus and/or Paracoccus.

The invention also provides a method of categorising skin type into a low level of age, a high level of age, or an intermediate level of age, wherein the method comprises determining the percentage abundance, abundance, or presence of bacteria from each of the following genera in a skin microbial sample:
a) Eikenella, Brochothrix Finegoldia, Lawsonella and Peptoniphilus; optionally wherein
   the genera predictive of a high level of age comprises any one or more or all of: Eikenella and/or Brochothrix; and
   the genera predictive of a low level of age comprises any one or more or all of: Finegoldia, Lawsonella and/or Peptoniphilus
b) Eikenella, Aerococcus, Glutamicibacter Finegoldia and Lawsonella; optionally wherein
   the genera predictive of a high level of age comprises any one or more or all of: Eikenella, Aerococcus and/or Glutamicibacter; and
   the genera predictive of a low level of age comprises any one or more or all of: Finegoldia and/or Lawsonella;
c) Aerococcus, Atopobium, Bacillus, Brachybacterium, Brochothrix, Chryseobacterium, Eikenella, Eubacterium, Glutamicibacter Pantoea, Bergeyella, Cloacibacterium, Finegoldia, Lawsonella, Peptoniphilus and Undibacterium; optionally wherein
   the genera predictive of a high level of age comprises any one or more or all of: Aerococcus, Atopobium, Bacillus, Brachybacterium, Brochothrix, Chryseobacterium, Eikenella, Eubacterium, Glutamicibacter and/or Pantoea; and
   the genera predictive of a low level of age comprises any one or more or all of: Bergeyella, Cloacibacterium, Finegoldia, Lawsonella, Peptoniphilus and/or Undibacterium.

The invention further provides a method of categorising skin type into a low level of barrier function, a high level of barrier function, or an intermediate level of barrier function, wherein the method comprises determining the percentage abundance, abundance, or presence of bacteria from each of the following genera in a skin microbial sample:
a) Cutibacterium, Paracoccus and Brevundimonas; optionally wherein
   the genera predictive of a low level of barrier function comprises any one or more or all of: Cutibacterium; and
   the genera predictive of a high level of barrier function comprises any one or more or all of: Paracoccus and/or Brevundimonas;
b) Eikenella, Cutibacterium, Micrococcus, Paracoccus, Brevundimonas, Bacillus and Xanthomonas; optionally wherein
   the genera predictive of a low level of barrier function comprises any one or more or all of: Eikenella and/or Cutibacterium; and
   the genera predictive of a high level of barrier function comprises any one or more or all of: Micrococcus, Paracoccus, Brevundimonas, Bacillus and/or Xanthomonas;
c) Eikenella, Cutibacterium Enhydrobacter, Micrococcus, Brevundimonas, Paracoccus and Bacillus; optionally wherein
   the genera predictive of a low level of barrier function comprises any one or more or all of: Eikenella and/or Cutibacterium; and
   the genera predictive of a high level of barrier function comprises any one or more or all of: Enhydrobacter, Micrococcus, Brevundimonas, Paracoccus and/or Bacillus;
d) Brochothrix, Cutibacterium, Eikenella, Lactobacillus, Turicella, Veillonella Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and Xanthomonas; optionally wherein
   the genera predictive of a low level of barrier function comprises any one or more or all of: Brochothrix, Cutibacterium, Eikenella, Lactobacillus, Turicella and/or Veillonella; and
   the genera predictive of a high level of barrier function comprises any one or more or all of: Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and/or Xanthomonas.

The invention further provides a method of categorising skin type into a low level of aged skin linked to smoking, a high level of aged skin linked to smoking, or an intermediate level of aged skin linked to smoking, wherein the method comprises determining the percentage abundance, abundance, or presence of bacteria from each of the following genera in a skin microbial sample:
a) Micrococcus, Bacillus, Eikenella, Gemella, Corynebacterium, Finegoldia, Cutibacterium and/or Anaerococcus.

In some embodiments of these methods, determining the percentage abundance of, abundance, or presence of each genera comprises sequencing a region of the microbial genome, optionally bacterial genome. Preferences for sequencing are described elsewhere and include Illumina sequencing, optionally using a MiSeq device, or by nanopore sequencing technology, optionally of the 16S rRNA variable region, optionally the V3V4 variable region.

Preferences for other features of the methods are described elsewhere, for example sample type and methods of obtaining the sample.

As described above, in some instances the microbial signature comprises threshold ratios to which the test sample must be compared. In some embodiments of these methods then the method comprises determining the ratio of total abundance of all genera associated with a high level of the characteristic to the total abundance of all genera associated with a low level of the characteristic.

In some embodiments, the skin microbial samples have been obtained by swabbing the skin, preferably from the face skin of the subject, such as from the cheek.

The methods described herein allow a prediction to be made as to whether the subject has skin that has a high or low level of a particular characteristic. Based on this prediction, it is possible to advise the use of particular cosmetic treatments, for example moisturises, that are considered to be able to address the characteristic if it is found to be disadvantageously too high or too low, for example a low level of hydration, of a high level of sebum. It would be advantageous to increase the level of hydration and reduce the level of sebum. Which characteristics are considered to be disadvantageous and require modulation are known to the skilled person.

Accordingly, the invention also provides a method of cosmetic treatment, comprising predicting whether a subject has a high level of a first skin characteristic or a low level of the first skin characteristic according to the methods described herein, and treating the skin with a cosmetic selected to improve the condition of the first skin characteristic.

The appropriate cosmetics will be known to the skilled person. The following is a non-exhaustive list:

### —To Improve the Age of the Skin:

Actives with a Proven Activity on the Ageing Signs & on the Skin Microbiome:
Agefinity™ (Glycerin (and) Water (and) Sodium Mannose Phosphate (and) Mannose); Brightenyl® (Water (and) Glycerin (and) Diglucosyl Gallic Acid); Revivyl™ (Propanediol (and) Orobanche Rapum Extract); Synchronight™ (Glycerin (and) Betaine (and) Water (and) Gardenia Jasminoides Fruit Extract (and) Maltodextrin); Vetivyne™ (Vetiveria Zizanoides Root Extract (and) Propanediol (and) Water); Yogurtene® Balance (Inulin (and) Yogurt Powder)
   Actives with a Proven Activity on Ageing Signs:
BlurHD® (Dipropylene Glycol (and) Gardenia Florida Fruit Extract); Commipheroline® (Caprylic/Capric Triglyceride (and) Commiphora mukul Resin Extract); Depollutine® (Water (and) Arginine PCA (and) Phaeodactylum tricornutum Extract); Easyliance® 2.0 (Acacia Senegal Gum (and) Rhizobian Gum); Eau de Source Marine (Spring Sea Water) (Sea water); Eau Vitale™ d'Algue Bleue (Water (and) Spirulina platensis Extract) Ellagi-C™ (Propanediol (and) Anogeissus LeiocarpA Bark Extract); Hyalusphere® (Sodium Hyaluronate Encapsulated); Hydrintense™ (Sea Water (and) Propanediol (and) Porphyridium Cruentum Extract) Inoveol® CAFA (Water (and) Caffeyl Glucoside) Inoveol® EGCG (Water (and) Epigallocatechin Gallatyl Glucoside); Inoveol® OLEU (Water (and) Oleuropeinyl Glucoside); Megassane® (Caprylic/Capric Triglyceride (and) Phaeodactylum tricornutum Extract; Neodermyl™ (Gylcerin (and) Water (and) Methylglucoside Phosphate (and) Copper Lysinate/Prolinate); NovHyal™ Biotech G (Glycerin (and) Water (and) Disodium Acetyl Glucosamine Phosphate); PrimalHyal 3K (Hydrolyzed hyaluronic acid); PrimalHyal™ 50 (Hydrolyzed Hyaluronic Acid); PrimalHyal™ Gold (Water (and) PEG-8 Caprylic/Capric Glycerides (and) Sodium Hyaluronate (and) Octyldodeceth-25); PrimalHyal™ Ultrafiller (Sodium Acetylated Hyaluronate); Redens'In™ (Commiphora mukul Resin Extract (and) Sodium Hyaluronate Encapsulated); Rosmarinyl™ Glucoside (Water (and) Rosmarinyl Glucoside) Rubixyl® (Water (and) Glycerin (and) Hexapeptide-48 HCL)
Softalia® (Fusanus Spicatus Kernel Oil); StimulHyal® (Calcium Ketogluconate); Tightenyl™ (Glycerin (and) Water (and) Disodium Acetyl Glucosamine Phosphate (and) Sodium Glucuronate (and) Magnesium Sulfate); Unichondrin™ ATP (Water (and) Butylene Glycol (and) Hydrolyzed Vegetable Protein (and) Adenosine Triphosphate (and) Sodium Chondroitin Sulfate); Unilactamin™ L-17 (Water (and) Butylene Glycol (and) Adenosine Triphosphate (and) Hydrolyzed Milk Protein (and) Niacinamide); Unilucent™ HR-14 (Water (and) Haberlea Rhodopensis Leaf Extract); Unilucent™ PA-13 (Panthenyl Triacetate (and) Acetyl Rheum rhaponticum Root Extract); Uniprosyn® PS-18 (Water (and) Butylene Glycol (and) Niacinamide (and) Adenosine Triphosphate (and) Hydrolyzed Oat Protein); Uniprotect® PT-3 (Panthenyl Triacetate (and) Ethyl Linoleate (and) Oleyl Alcohol (and) Tocopherol); Unirepair® T-43 (Water (and) Butylene Glycol (and) Acetyl Tyrosine (and) Proline (and) Hydrolyzed Vegetable Protein (and) Adenosine Triphosphate); Unisteron™ Y-50 (Oleyl Alcohol (and) Dioscorea villosa (Wild Yam) Root Extract (and) Hexyldecanol (and) Glycine soja (Soybean) Sterols); Unisurrection™ S-61 (Water (and) Beta vulgaris (Beet) Root Extract (and) Glycerin (and) Haberlea; Rhodopensis Leaf Extract (and) Yeast Extract); Yogurtene® (Yogurt powder).

### —To Improve the Sensitivity Status:

Actives with a Proven Activity on the Sensitive Status & on the Skin Microbiome:
Sensityl™ (Water (and) Phaeodactylum tricornutum Extract (and) Pentylene Glycol)
   Actives with a Proven Activity on the Sensitive Status:
Biogomm'age™ UE series (Cellulose (and) Hydroxypropylcellulose (and) Tocopheryl Acetate (and) CI77007); Biogomm'age™ WD series (Cellulose (and) Hydroxypropylcellulose (and) Panthenyl Triacetate), BisaboLife™ (Bisabolol); D-Panthenyl Triacetate (Panthenyl Triacetate); Endothelyol™ (Glycerin (and) Rosmarinyl Glucoside (and) Caffeyl Glucoside (and) Gallyl Glucoside); Erasyal™ (Glycerin (and) Disodium Acetyl (and) Glucosamine Phosphate (and) Caffeyl Glucoside); Grevilline™ PF (Glycerin (and) Water (and) Skeletonema Costatum extract); Inoveol® CAFA (Water (and) Caffeyl Glucoside); Inoveol® EGCG (Water (and) Epigallocatechin Gallatyl Glucoside) Inoveol® OLEU (Water (and) Oleuropeinyl Glucoside); Mariliance™ (Water (and) Propanediol (and) Rhodosorus Marinus Extract); Neurophroline™ (Water (and) Propanediol (and) Tephrosia purpurea Seed Extract); Ocâline™ (Sea Water (and) Water (and) Cucurbita pepo (Pumpkin) Seed Extract (and) Citric Acid); Rosmarinyl™ Glucoside (Water (and) Rosmarinyl Glucoside); Soothex® (Isostearyl Alcohol (and) Boswellia serrata Gum); Uniglucan™ G-51 (Water (and) Butylene Glycol (and) Yeast Polysaccharides); Uniprotect® PT-3 (Panthenyl Triacetate (and) Ethyl Linoleate (and) Oleyl Alcohol (and) Tocopherol); Unirepair® T-43 (Water (and) Butylene Glycol (and) Acetyl Tyrosine (and) Proline (and) Hydrolyzed Vegetable Protein (and) Adenosine Triphosphate); Unisooth™ EG-28 (Water (and) Propyl Gallate (and) Gallyl Glucoside (and) Epigallocatechin Gallatyl Glucoside); Unisooth™ PN-47 (Panthenyl Triacetate (and) Naringenin); Unisooth™ ST-32 (Water (and) Pentylene Glycol (and) Tamarindus Indica Seed Gum (and) Stevioside); Unispheres® Colour boosting (CI 77499 (and) Mannitol (and) Cellulose (and) Carapa Guaianensis Seed Oil (and) Hydroxypropyl Methylcellulose (and) CI 77891 (and) CI 77289); Unispheres® Flashtone G (Mannitol (and) Cellulose (and) Barium Sulfate (and) Acrylates Copolymer (and) Silica (and) Hydroxypropyl Methylcellulose)
   —To Improve the Skin Barrier Function (the Recommendation would have been the Same in Case of LOW Hydration Profile):
   Actives with a Proven Activity on the Barrier Function & on the Skin Microbiome:
Agefinity™ (Glycerin (and) Water (and) Sodium Mannose Phosphate (and) Mannose)
Brightenyl® (Water (and) Glycerin (and) Diglucosyl Gallic Acid)
Sensityl™ (Water (and) Phaeodactylum tricornutum Extract (and) Pentylene Glycol)
Vetivyne™ (Vetiveria Zizanoides Root Extract (and) Propanediol (and) Water)
Yogurtene® Balance (Inulin (and) Yogurt Powder)
   Actives with a Proven Activity on the Barrier Function:
Abdoliance™ (Paullinia Cupana Seed Extract (and) Hesperetin Encapsulated)
Appygreen™ 812 (Water (and) Decyl Glucoside (and) Xylose (and) Decyl Alcohol)
Axolight® (Water (and) Hydrolyzed Wheat Flour), B-Lightyl
Bamboosilk (Bambusa arundinacea stem powder); Biogomm'age™ UE series (Cellulose (and) Hydroxypropylcellulose (and) Tocopheryl Acetate (and) CI77007) Biogomm'age™ WD series (Cellulose (and) Hydroxypropylcellulose (and) Panthenyl Triacetate); BisaboLife™ (Bisabolol); BlurHD® (Dipropylene Glycol (and) Gardenia Florida Fruit Extract); Ceramide II/Uniblend™ 2G (Ceramide NG, Water (and) PEG-7 Glyceryl Cocoate (and) PEG 60 Hydrogenated Castor Oil (and) Ceramide NG (and) Gluconolactone); Commipheroline® (Caprylic/Capric Triglyceride (and) Commiphora mukul Resin Extract); Cristalhyal® Range (Sodium Hyaluronate); D-Panthenyl Triacetate (Panthenyl Triacetate); Depollutine® (Water (and) Arginine PCA (and) Phaeodactylum tricornutum Extract); Easyliance® 2.0 (Acacia Senegal Gum (and) Rhizobian Gum) Eau de Source Marine (Spring Sea Water) Sea water; Eau Vitale™ d′Algue Bleue (Water (and) Spirulina platensis Extract); Ellagi-C™ (Propanediol (and) Anogeissus leiocarpA Bark Extract); Endothelyol™ (Glycerin (and) Rosmarinyl Glucoside (and) Caffeyl Glucoside (and) Gallyl Glucoside); Erasyal™ (Glycerin (and) Disodium Acetyl (and) Glucosamine Phosphate (and) Caffeyl Glucoside)
Evercool® Skin (Menthyl PCA (and) Lactamide MEA (and) Menthane Carboxamide Ethylpyridine); Exfoliance Range (Prunus armeniaca (Apricot) Seed Powder, Oryza sativa (Rice) Hull Powder); Glossyliance™ (Water (and) Saccharum officinarum (Sugar Cane) Extract (and) Citrus limon (Lemon) Peel Extract (and) Lactic Acid (and) Malic Acid (and) Citric Acid); Grevilline™ PF (Glycerin (and) Water (and) Skeletonema Costatum extract) Hairsphere; Hyalusphere® (Sodium Hyaluronate Encapsulated); Hydreïs™ (Water (and) Hydrolyzed Beta-Glucan); Hydrintense™ (Sea Water (and) Propanediol (and) Porphyridium Cruentum Extract); Inoveol® CAFA (Water (and) Caffeyl Glucoside) Inoveol® EGCG (Water (and) Epigallocatechin Gallatyl Glucoside); Inoveol® OLEU (Water (and) Oleuropeinyl Glucoside); K-phyto [SC] Camellia K-phyto [PP] GHK; Karanja Oil (Pongamia glabra Seed Oil); Kendi oil (Aleurites moluccanus Seed Oil): Lipsphere; Lithocosmetics (Tourmaline, Nephrite Powder, Sapphire powder, Rub; Mariliance™ (Water (and) Propanediol (and) Rhodosorus Marinus Extract) Megassane® (Caprylic/Capric Triglyceride (and) Phaeodactylum tricornutum Extract) Muciliance® Fruit (Galactaric acid); Neodermyl™ (Gylcerin (and) Water (and) Methylglucoside Phosphate (and) Copper Lysinate/Prolinate)
Neurophroline™ (Water (and) Propanediol (and) Tephrosia purpurea Seed Extract) NovHyal™ Biotech G (Glycerin (and) Water (and) Disodium Acetyl Glucosamine Phosphate); Nyamplung oil (Calophyllum Inophyllum Seed Oil); Ocâline™
   (Sea Water (and) Water (and) Cucurbita pepo (Pumpkin) Seed Extract (and) Citric Acid); Pongamia Extract Pongamia glabra Seed Oil; PrimalHyal 3K Hydrolyzed hyaluronic acid; PrimalHyal™ 300 Hydrolyzed Hyaluronic Acid
PrimalHyal™ 50 Hydrolyzed Hyaluronic Acid
PrimalHyal™ GoldWater (and) PEG-8 Caprylic/Capric Glycerides (and) Sodium Hyaluronate (and) Octyldodeceth-25
PrimalHyal™ Ultrafiller; Pro-DG™Water (and) Dysmorphococcus Globosus Extract; Questice® Range “Questice Liquid: Menthyl PCA; Questice Plus: Menthyl PCA (and) Menthol (and) Dipropylene Glycol”; Redensyl™ Water (and) Glycerin (and) Sodium Metabisulfite (and) Glycine (and) Larix europaea Wood Extract (and) Zinc Chloride (and) Camellia sinensis Leaf Extract; Redens'In™ Commiphora mukul Resin Extract (and) Sodium Hyaluronate Encapsulated; ResistHyal™ Water (and) Hydrolysed Hyaluronic Acid (and) Sodium Hyaluronate
Revivyl™ Propanediol (and) Orobanche Rapum Extract; Rosmarinyl™ Glucoside Water (and) Rosmarinyl Glucoside; Rubixyl® Water (and) Glycerin (and) Hexapeptide-48 HCL Safester™ A-75 Ethyl Linoleate; Sens'Hyal™Water (and) Rhizobian Gum (and) Sodium Hyaluronate; Silkalun™ Potassium Alum (and) Magnesium Stearate; Sinodor® Citronellyl Methylcrotonate; Softalia® Fusanus Spicatus Kernel Oil; Softolive™Hydrogenated Ethylhexyl Olivate (and) Hydrogenated Olive Oil Unsaponifiables
Soligel™ Rhizobian Gum; Soothex® Isostearyl Alcohol (and) Boswellia serrata Gum; Sophogreen™ PlusWater (and) Hydrolysed Candida bombicola Extract; Sopholiance™ S
   Candida bombicola (and) Glucose (and) Methyl Rapeseedate Ferment)
StimulHyal® Calcium Ketogluconate; Synchronight; Syner-GX™ (Cyamopsis tetragonoloba) Tightenyl™; Unichondrin™ ATPWater (and) Butylene Glycol (and) Hydrolyzed Vegetable Protein (and) Adenosine Triphosphate (and) Sodium Chondroitin Sulfate
Unicontrozon™ C-49Water (and) Propylene Glycol (and) Citrus Lemon (Lemon) Extract (and) Fumaria officinalis Flower/Leaf/Stem Extract (and) Fumaric Acid Uniglucan™ G-51Water (and) Butylene Glycol (and) Yeast Polysaccharides Unilactamin™ L-17Water (and) Butylene Glycol (and) Adenosine Triphosphate (and) Hydrolyzed Milk Protein (and) Niacinamide; Unilucent™ HR-14 Water (and) Haberlea Rhodopensis Leaf Extract
Unilucent™ PA-13Panthenyl Triacetate (and) Acetyl Rheum rhaponticum Root Extract Unimer U-6 Triacontanyl PVP; Unimer U-15 VP/Eicosene Copolymer; Unimer U-151 VP/Hexadecene Copolymer; Unimer U-1946 VP/Hexadecene Copolymer (and) Octyldodecanol; Unimoist™ U-125 GWater (and) Glycerin (and) Urea (and) Saccharide Hydrolysate (and) Magnesium Aspartate (and) Glycine (and) Alanine (and) Creatine Uninontan™ U-34Water (and) Propylene Glycol (and) Sodium Citrate (and) Citrus Limon (Lemon) Fruit Extract (and) Cucumis sativus (Cucumber) Fruit Extract Unipertan™ Water (and) Butylene Glycol (and) Acetyl Tyrosine (and) Hydrolyzed Vegetable Protein (and) Riboflavin Adenosine Triphosphate; Uniprosyn® PS-18 (Hydrolyzed Oat Protein); Uniprotect® PT-3Panthenyl Triacetate (and) Ethyl Linoleate (and) Oleyl Alcohol (and) Tocopherol; Unireduce®; Unirepair®; Unishapes; Unisooth™ Unisooth™ PN-47; Unisooth™ ST-32; Unispheres® Range; Unispheres® Bicolour Unispheres® Flashtone GMannitol (and) Cellulose (and) Barium Sulfate (and) Acrylates Copolymer (and) Silica (and) Hydroxypropyl Methylcellulose
Unispheres® Flashwhite
Unisteron™ Y-50Oleyl Alcohol (and) Dioscorea villosa (Wild Yam) Root Extract (and) Hexyldecanol (and) Glycine soja (Soybean) Sterols
Unisurrection™ S-61Water (and) Beta vulgaris (Beet) Root Extract (and) Glycerin (and) Haberlea Rhodopensis Leaf Extract (and) Yeast Extract
Unitamuron™ H-22Water (and) Pentylene Glycol (and) Tamarindus Indica Seed Gum
Unititamer™ T-40Titanium Dioxide (and) Triacontanyl PVP (and) Isoeicosane
Unitrienol™ T-27Farnesyl Acetate (and) Farnesol (and) Panthenyl Triacetate
Uvaxine™ Water (and) Glycerin (and) Polydatin Glucoside
Vegetan® Dihydroxyacetone; Vegetan® FL Dihydroxyacetone (and) Water; Vegetan® FL CreationDihydroxyacetone (and) Water (and) Parfum (and) Polysorbate 20 (and) Caprylyl/Capryl Wheat Bran/Straw Glycosides; Vegetan® Gold; Vegetan® Life Vitasphere; Wakamine™ Water (and) Undaria Pinnatifida Extract
Whitesphere™ Glycyrrhiza glabra (licorice) Root Extract (and) Magnesium Ascorbyl Phosphate (and) Undaria Pinnatifida Extract Encapsulated
Xyliance™ Cetearyl Wheat Straw Glycosides (and) Cetearyl Alcohol; Yogurtene® Yogurt powder

### —To Reduce the Sebum Quantity:

Actives with a Proven Activity on the Sebum Regulation & on the Skin Microbiome:
Brightenyl® (Water (and) Glycerin (and) Diglucosyl Gallic Acid)
Sopholiance™ S (Candida bombicola (and) Glucose (and) Methyl Rapeseedate Ferment (and) Water)
   Actives with a Proven Activity on the Sebum Regulation:
Bamboosilk (Bambusa arundinacea stem powder); Erasyal™ (Glycerin (and) Disodium Acetyl (and) Glucosamine Phosphate (and) Caffeyl Glucoside); K-phyto [PP] GHK (Water (and) Caffeoyl Tripeptide-1); PrimalHyal™ 300 (Hydrolyzed Hyaluronic Acid)
Safester™ A-75 (Ethyl Linoleate); Unireduce® R-35 (Farnesyl Acetate (and) Panthenyl Triacetate (and) Tocopheryl Acetate); Unisteron™ Y-50 (Oleyl Alcohol (and) Dioscorea villosa (Wild Yam) Root Extract (and) Hexyldecanol (and) Glycine soja (Soybean) Sterols); Unitrienol™ T-27 (Farnesyl Acetate (and) Farnesol (and) Panthenyl Triacetate)

### —To Improve Dark Spots Pre-Disposition:

Actives with a Proven Activity on the Dark Spot Modulation & on the Skin Microbiome:
Agefinity™ (Glycerin (and) Water (and) Sodium Mannose Phosphate (and) Mannose)
Brightenyl® (Water (and) Glycerin (and) Diglucosyl Gallic Acid)
   Actives with a Proven Activity on the Dark Spot Modulation:
Axolight® (Water (and) Hydrolyzed Wheat Flour); B-Lightyl™ (Himanthalia Elongata Extract)
Ellagi-C™ (Anogeissus leiocarpA Bark Extract); Megassane® (Caprylic/Capric Triglyceride (and) Phaeodactylum tricornutum Extract); Neurophroline™ (Tephrosia purpurea Seed Extract); Unilucent™ HR-14 (Haberlea Rhodopensis Leaf Extract)
Unilucent™ PA-13 (Acetyl Rheum rhaponticum Root Extract); Uninontan™ U-34 (Citrus Limon (Lemon) Fruit Extract (and) Cucumis sativus (Cucumber) Fruit Extract)
Unispheres® Flashwhite (Citrus Limon (Lemon) Fruit Extract (and) Cucumis sativus (Cucumber) Fruit Extract (and) Caprylic/Capric Triglyceride (and) Glyceryl Stearate Citrate (and) Decyl Glucoside (and) Hydroxypropyl Methylcellulose); Wakamine™ (Water (and) Undaria Pinnatifida Extract); Whitesphere™ (Glycyrrhiza glabra (licorice) Root Extract (and) Magnesium Ascorbyl Phosphate (and) Undaria Pinnatifida Extract Encapsulated)

Since the methods described herein are able to demonstrate which microbes, for example which bacterial genera are associated with a high or a low level of a characteristic, it is possible to monitor any changes in the microbial profile to determine if a cosmetic treatment is having a desired effect. For example it is possible to monitor whether the microbial profile of a low hydrated skin moves towards that of a high hydrated skin following cosmetic treatment.

Accordingly, the invention provides a method of monitoring the efficacy of a cosmetic treatment, comprising:
(i) providing a first test skin microbial sample taken from a subject and predicting whether the subject has a high level of at least a first skin characteristic, a low level of at least a first skin characteristic, or an intermediate level of at least a first skin characteristic according to the method of any of the embodiments described previously;
(ii) treating the skin with a cosmetic selected to modulate the level of the at least one characteristic; and
(iii) providing a second test skin microbial sample taken from a subject and predicting whether the subject has a high level of at least a first skin characteristic, a low level of at least a first skin characteristic, or an intermediate level of at least a first skin characteristic according to the method of any of the embodiments described previously;
(iv) determining whether the level of the at least a first skin characteristic has been desirably modulated.

Preferences for the features of the method, for example skin characteristic, are as described elsewhere.

The invention also provides a method of preparing a data carrier containing data on at least one predicted characteristic of human skin type, the method comprising carrying out the method of any one of the embodiments described previously and recording the results on a data carrier.

The invention also provides a kit of parts comprising oligonucleotides designed to amplify a unique region of the genome of any one or more of the combinations of bacterial genera of any of the embodiments described previously.

The invention also provides a kit of parts comprising a set of probes designed to detect the presence and/or abundance of any one or more of the combinations of bacterial genera of any of any of the embodiments described previously.

The invention also provides a portable device capable of detecting the percentage abundance, abundance, or presence of any one or more of the combinations of bacterial genera of any of any of the embodiments described previously. In some embodiments the device is capable of taking a microbial sample from the skin of the subject. In some embodiments the device is capable of comparing the percentage abundance, abundance, or presence of a combination of bacterial genera in a test sample with a pre-determined microbial signature, and producing a prediction as to whether the subject has a high level of a first skin characteristic, an intermediate level of the first skin characteristic, or a low level of the first skin characteristic. In some embodiments, the device displays suggested cosmetic treatments based on the prediction.

The invention provides a microbial signature as described herein or as determined by any of the methods of the invention.

The invention also provides a cosmetic composition comprising any one or more of the following combinations of bacterial genera:
a) Haemophilus, Kocuria and/or Neisseria;
b) Haemophilus, Kocuria, Neisseria and/or Paucibacter;
c) Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and/or Brochothix;
d) Cutibacterium;
e) Cutibacterium and/or Staphylococcus;
f) Bacillus, Cutibacterium, Dolosigranulum, Eikenella, Staphylococcus and/or Stenotrophomonas;
g) Bacillus, Campylobacter, Cutibacterium, Delftia, Dolosigranulum, Eikenella, Frigoribacterium, Glutamicibacter, Moraxella, Serratia, Staphylococcus, Stenotrophomonas and/or Undibacterium;
h) Haemophilus and/or Snodgrassella;
i) Bacillus, Haemophilus and/or Kocuria;
j) Bacillus, Haemophilus, Kocuria, Paucibacter and/or Snodgrassella;
k) Allorhizobium, Amaricoccus, Bacillus, Brevundimonas, Falsirhodobacter, Haemophilus, Kocuria, Lautropia, Paucibacter and/or Snodgrassella;
l) Micrococcus and/or Paracoccus;
m) Kocuria, Micrococcus, Paracoccus and/or Bergeyella;
n) Alloiococcus, Bergeyella, Exiguobacterium, Kocuria, Micrococcus and/or Paracoccus;
o) Eikenella and/or Brochothrix;
p) Eikenella, Aerococcus and/or Glutamicibacter;
q) Aerococcus, Atopobium, Bacillus, Brachybacterium, Brochothrix, Chryseobacterium, Eikenella, Eubacterium, Glutamicibacter and/or Pantoea;
r) Cutibacterium;
s) Eikenella and/or Cutibacterium; and/or
t) Brochothrix, Cutibacterium, Eikenella, Lactobacillus, Turicella and/or Veillonella.

Combinations (a) to (c) are considered to aid in lowering sebum levels, and so are considered to be useful for subjects identified by the present methods as having a high level of sebum.

Combinations (d) to (g) are considered to aid in lowering sensitivity levels, and so are considered to be useful for subjects identified by the present methods as having a high level of sensitivity.

Combinations (h) to (k) are considered to aid in increasing hydration levels, and so are considered to be useful for subjects identified by the present methods as having a low level of hydration.

Combinations (l) to (n) are considered to aid in lowering brown spot levels, and so are considered to be useful for subjects identified by the present methods as having a high levels of brown spots.

Combinations (o) to (q) are considered to aid in lowering the apparent age level of the skin, and so are considered to be useful for subjects identified by the present methods as having aged or prematurely aged skin (i.e. a high level of age, or a higher level of age compared to true age).

Combinations (r) to (t) are considered to aid in increasing skin barrier levels, and so are considered to be useful for subjects identified by the present methods as having a low level of skin barrier function.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention.

### FIGURE LEGENDS

In the figures, LOW in the context of TEWL level indicates a high level of barrier function, and HIGH in the context of TEWL level indicates a low level of barrier function.

FIG. 1—Microbial profile of skin with different characteristics. Genera shown in shaded bold are those associated with a high level of the characteristic (e.g. high level of sebum) and those in non-bold no shading are genera associated with a low level of the same condition. A) Microorganisms relevant for sebum level assessment. B) Microorganisms relevant for age assessment. C) Microorganisms relevant for sensitivity assessment. D) Microorganisms relevant for spotting level assessment. E) Microorganisms relevant for Hydration level assessment. F) Microorganisms relevant for TEWL assessment-LOW in the context of TEWL level indicates a high level of barrier function, and HIGH in the context of TEWL level indicates a low level of barrier function.

FIG. 2—Bacteria identified with the foldchanges approach above are used to perform a Random Forest analysis. The figure shows relevant bacteria to define each skin type signatures in using Illumina MiSeq sequencing. A) Microorganisms relevant for sebum level assessment. B) Microorganisms relevant for age assessment. C) Microorganisms relevant for sensitivity assessment. D) Microorganisms relevant for spotting level assessment. E) Microorganisms relevant for hydration level assessment. F) Microorganisms relevant for TEWL assessment-LOW in the context of TEWL level indicates a high level of barrier function, and HIGH in the context of TEWL level indicates a low level of barrier function.

FIG. 3—Shows the major relevant bacteria to define skin types in using Illumina MiSeq sequencing and the accuracy associated the signature of each skin parameter.

FIG. 4—Shows relevant bacteria to define each skin type signatures in using Illumina MiSeq sequencing in limiting the signature to genera also detected with Nanopore sequencing technology. Microorganisms relevant for sebum level assessment. Microorganisms relevant for age assessment. Microorganisms relevant for sensitivity assessment. Microorganisms relevant for spotting level assessment. Microorganisms relevant for hydration level assessment. Microorganisms relevant for TEWL assessment-LOW in the context of TEWL level indicates a high level of barrier function, and HIGH in the context of TEWL level indicates a low level of barrier function.

FIG. 5—shows the relevant bacteria to define skin types and the accuracy associated the signature of each skin parameter in using Illumina MiSeq sequencing in limiting the signature to genera also detected with Nanopore sequencing technology.

FIG. 6—Taxonomy assignation of Nanopore reads of volunteer A.

FIG. 7—Taxonomy assignation of Nanopore reads of volunteer A.

### EXAMPLES

### Example 1—Methods

Samples were processed as according to PCT application PCT/EP20201075530, and as described below.

To define microbiota signatures, a swabbing was performed on the cheeks of Caucasian women reporting different ages, skin sensitivity, hydration, sebum and dark spots levels. A minimum of 17 volunteers were included per group (low hydration level group, high sebum level group). A written informed consent was obtained before the start of the study. On arrival, volunteers were put in a controlled environment (20° C., relative humidity of 45-50%) for a minimum period of 10 minutes before sample collection.

Groups were designed as no confounding factor could be considered between two groups opposed by a same parameter, limiting the differences between groups at the property studied. In considering the example of the “high skin sensitivity group” versus “the normal sensitivity group”, there are no significant differences in term of age, hydration, sebum and spots levels between these two groups; the only significant difference is the level of skin sensitivity (significant difference means having a pvalue<0.05*). To assess the statistical difference, a t-test has been used.

The sebum level is measured using a Sebumeter®. It is a photometric method. A synthetic ribbon, which becomes transparent when in contact with absorbed lipids, is applied to the measurement zone for precisely 30 seconds. Its transparency increases proportionally with the quantity of sebum from the hydrolipidic film with which it is in contact. A reflectometry recording is used to quantify the increase of the light transmitted and to determine the total mass of the lipids excreted by the surface unit (in μg/cm²).

The hydration level is measured using a Corneometer®. The stratum corneum hydration causes changing in its electrical characteristics. The stratum corneum is like a dielectric corps. Any modifications of its hydration statement cause a variation of the electric capacity measured by a condenser. Higher is the hydration, higher is the electric capacity because its dipolar nature increases the electric permittivity of the environment and its conductibility. Measurement was performed by the Corneometer® CM 825 TM (Courage & Khazaka electronics). The probe linked to a condenser allows applying at all the time the same pressure on the tegument in order to not disturb the measures and obtain good experimental conditions reproducibility.

The Trans-Epidermal Water Loss (TEWL) is measured using a Tewameter®. TEWL is the passive diffusion of water through the stratum corneum from the inside of the body to the outside. Its measurement makes it possible to appreciate the integrity of the barrier function. A probe delimiting a cylindrical chamber in contact with the skin is used to measure the gradient of water vapor established on the cutaneous surface, an open chamber in order to partially overcome variations in environmental conditions. The unit of measurement is g/m²/h.

The pH level is measured using a pHmeter®. The pH 905 probe allows measuring the hydrogen potential. To do this, simply place the probe on the measuring zone and the measurement is given immediately.

The sensitive skin phenotype was established on the basis of the adverse sensory response to capsaicin test.

Brown spots (pigmentation and discoloration on and beneath the surface of the skin) is measured using a Visia CR 2.3® from Canfield® imaging systems. The VISIA allows taking pictures with different types of illuminations and a very rapid capture of images. A series of photos taken under multi-spectral imaging and analysis allow capturing visual information affecting appearance of the skin. Canfield's RBX® Technology separates the unique color signatures of Red and Brown skin components for unequaled visualization of conditions that result in color concentration, such as spider veins, hyperpigmentation, inflammation and other conditions. Because of the difference of the size of faces of the volunteers, a normalization of the number of spots has been made is using the surface considered for the measurement.

Results related to skin parameters measurement are shown in table 1.

TABLE 1
Measurements of skin parameters.

| | | Number | | | | | | |
|---|---|---|---|---|---|---|---|---|
| | | of | | Mean | | Mean | Mean | |
| | | volunteers per | | Hydration | Mean | Sebum | Spots | Mean |
| Group | Condition | group | Mean pH | level | TEWL | level | number | Age |
| Sebum | Low | 20 | 5.11 | 49.62 | 13.02 | 9.85* | Unknown | 34.4 |
| | High | 20 | 5.23 | 53.55 | 20.38 | 92.20* | Unknown | 31.8 |
| Age | Younger | 20 | 5.25 | 51.9 | 20.66 | 53.7 | Unknown | 21.9* |
| | Older | 20 | 5.15 | 50.7 | 14.61 | 39.65 | Unknown | 46.2* |
| Sensitivity | Non | 20 | 5.27 | 57.26 | 12.80 | 43.28 | Unknown | 30.8 |
| | sensitive | | | | | | | |
| | Sensitive | 17 | Unknown | Unknown | Unknown | Unknown | Unknown | 34.15 |
| Dark | Low | 19 | 5.18 | 52.87 | 14.25 | 50.32 | 285.89* | 29.32 |
| spots | High | 19 | 5.29 | 49.18 | 13.61 | 56.42 | 496.79* | 29.95 |
| Hydration | Low | 18 | 5.18 | 37.61* | 21.34 | 41.79 | Unknown | 33.7 |
| | High | 19 | 5.18 | 58.96* | 11.68 | 34.89 | Unknown | 34.0 |
| TEWL | Low | 19 | 5.29 | 53.92 | 10.20* | 38.15 | Unknown | 33.3 |
| | High | 20 | 5.19 | 45.63 | 25.57* | 52.76 | Unknown | 32.45 |

Skin bacteria were collected from the right and left cheeks (25 cm²/cheek). Sterile swabs moistened with a sterile solution of 0.15 M NaCl were used as a non-invasive swabbing method. All the samples were systematically sampled using a standardized procedure. Swabs were stored at −20° C. until DNA extraction. DNA extraction was performed using the DNeasy PowerLyzer PowerSoil DNA Isolation Kit (Qiagen, Germantown, MD, USA) with the following modifications. The tip of each swab was detached with a sterile surgical blade and transferred to a 1.5 ml tube containing 750 μL of Bead Solution. The sampled biomass was suspended by stirring and pipetting and then transferred to a bead beating tube. The remaining steps were performed according to the manufacturer's instructions. The DNA concentration was determined using the QuBit dsDNA HS fluorometric quantitation kit (Invitrogen, ThermoFisher Scientific, Courtaboeuf, France) according to the manufacturer's instructions.

DNA amplification and sequencing 16S rRNA gene sequencing was performed using a MiSeq device (Illumina, Inc., San Diego, CA, USA) through a 500 cycle paired-end run, as we previously reported. Briefly, the V3V4 16S rRNA variable regions were targeted. The global procedure involved two PCR steps. The libraries preparation and the MiSeq run were performed by Givaudan Active Beauty on the GeT-PlaGe platform (INRAe, Auzeville, France).

After the MiSeq run, raw data sequences were demultiplexed and quality-checked to remove all the reads with ambiguous bases. Indexes and primer sequences were removed with cutadapt (v1.9; http://cutadapt.readthedocs.io/en/stable/index.html) and reads with a fastq score lower than 28 were trimmed. The forward and reverse sequences were paired using bbmerge (https://jgi.doe.gov/data-and-tools/bbtools/). Samples with less than 5000 paired sequences were discarded. The remaining paired sequences were then treated us using an inhouse pipeline that uses vsearch to remove chimeras and amplicons with PCR errors. Sequences were then split into operational taxonomic units (OTUs, a cluster of similar sequence variants of the 16S rRNA marker gene sequence) at a 1% dissimilarity level using swarm (v2.6). Unique amplicons were mapped to the SILVA SSU Ref NR 99 (nonredundant) database (release 132; https://www.arb-silva.de/) for taxonomic assignation using the RDP classifier [20]. Data normalization and analyses were done using an R statistical computing environment (v3.2.0; https://www.r-project.org/) R core team (2014) using Bioconductor package (mainly Phyloseq, DESeq2 and Vegan libraries; http://www.bioconductor.org/).

Relevant microorganisms of each condition (pvalue<0.05) were obtained using the statistical package DESeq2 (Bioconductor) by comparing relative abundances of the taxa and the subsets of relevant microorganisms between each analysed condition (Sebum, Hydration, Age, Sensitivity, Dark spots). The padj represent the adjusted pvalue using multiple testings. The foldchange describes how much quantities are changing between a condition to another.

Microorganisms with a pvalue<0.05, a relative abundance higher than 0.1% and found in at least 30% of the samples were then classified using a machine learning approach based on a RandomForest classifier (RamdomForest package from R) to define more precisely which bacteria is the more relevant to characterize a specific skin condition. Quickly, the Random Forest approach uses thousands of phylogenetic trees to define which bacterial genera allow the better to discriminate samples according to the main variable (example of the variable: sebum level).

Accuracy of the predicted classifiers was validated using an external dataset and Area under curve (AUC) was used to determine accuracy of the model. The sensitivity and specificity of the classifier was obtained by comparing the table of predicted and classified individuals using a confusion matrix (Confusion matrix package from R).

### Example 2—Microorganisms Identified in Each Skin Condition

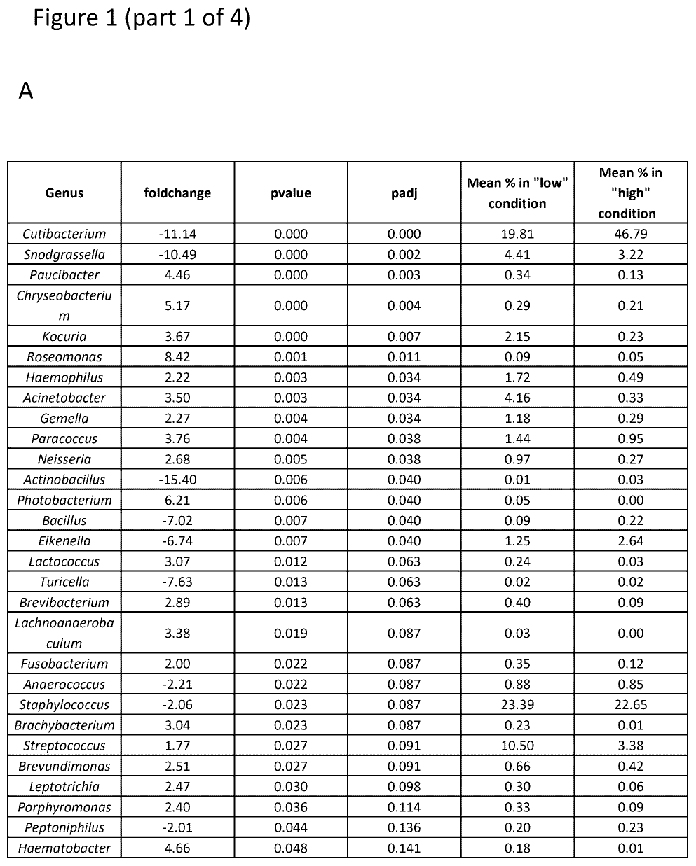

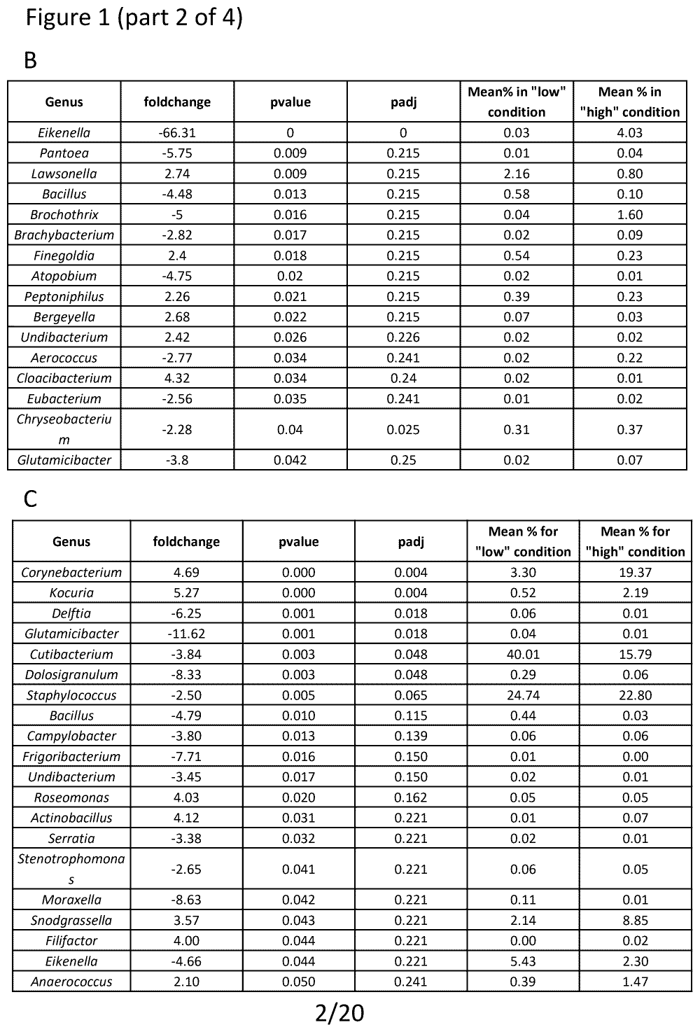

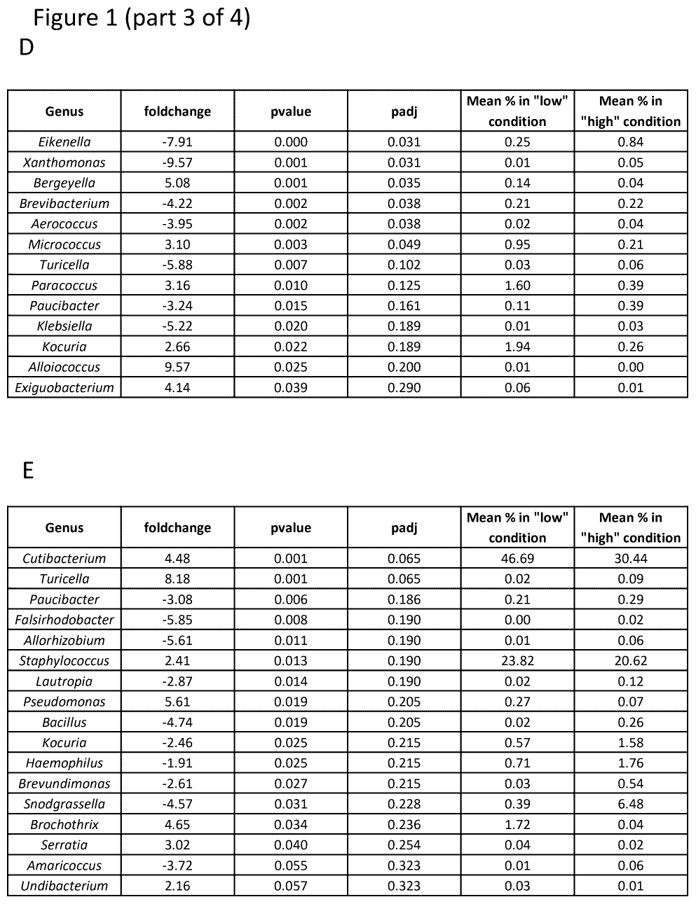

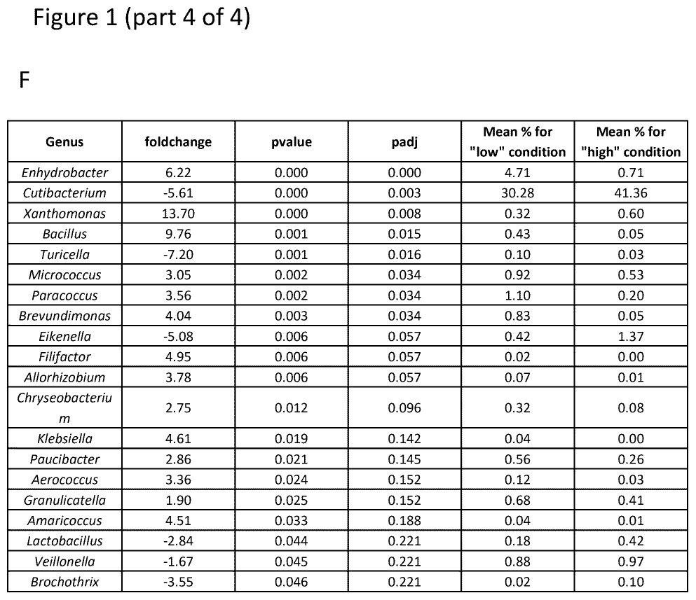

The microorganisms identified in each skin condition at shown in FIG. 1.

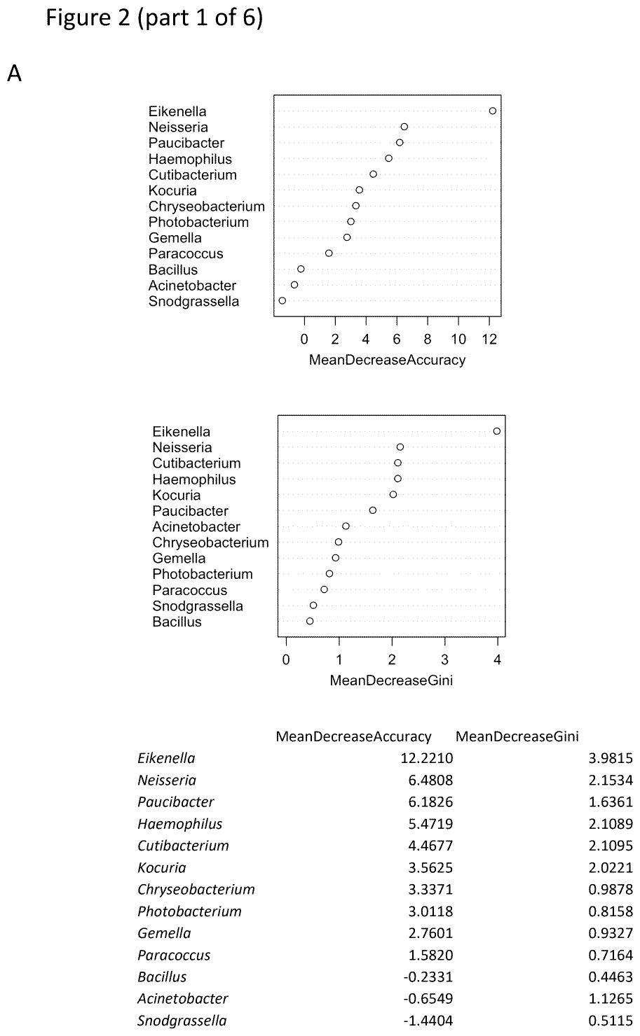

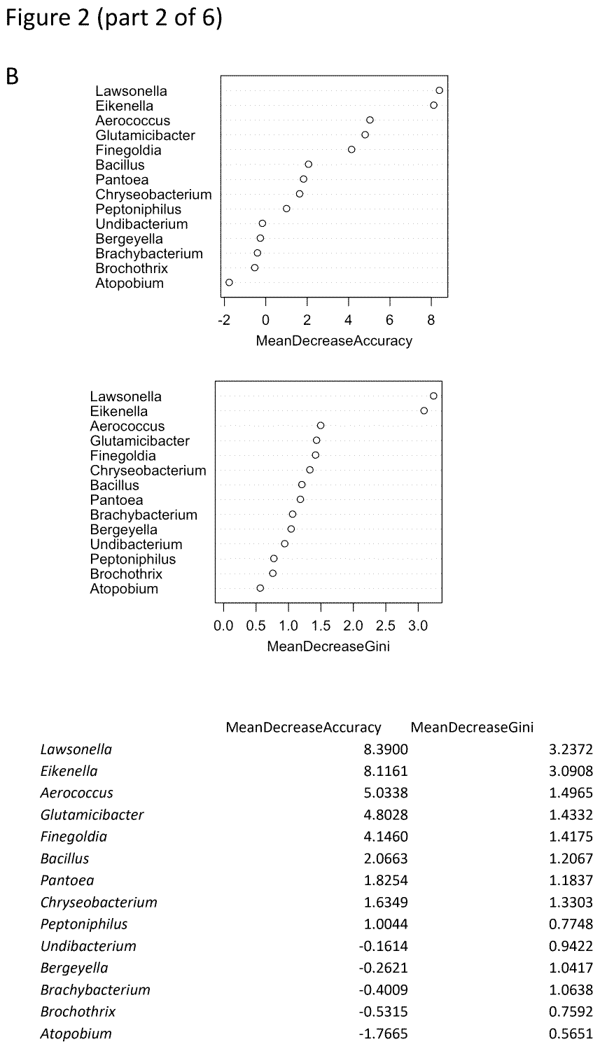

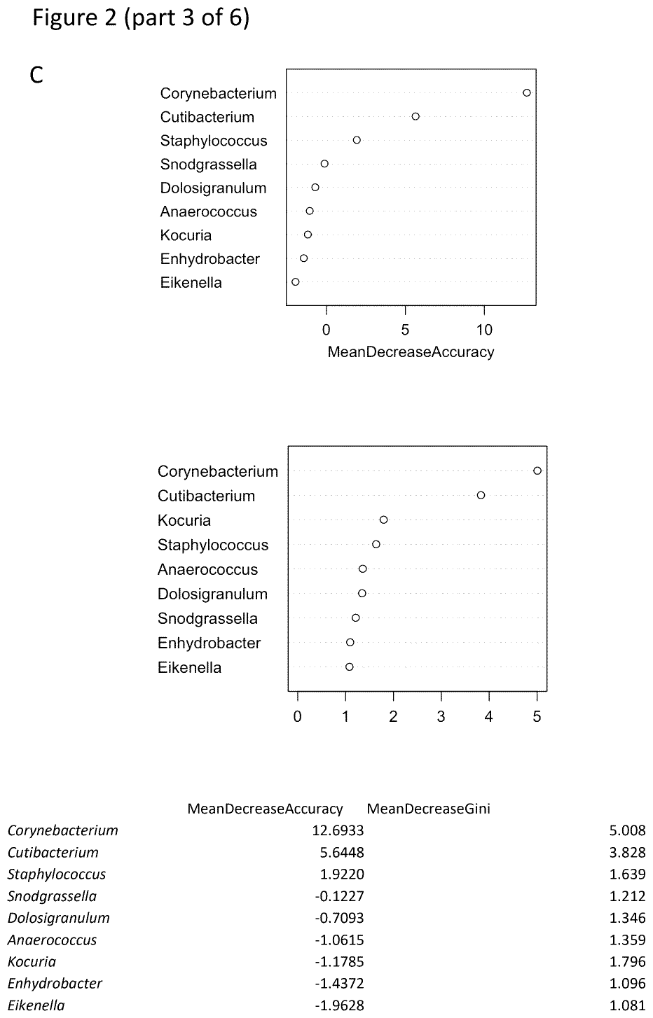

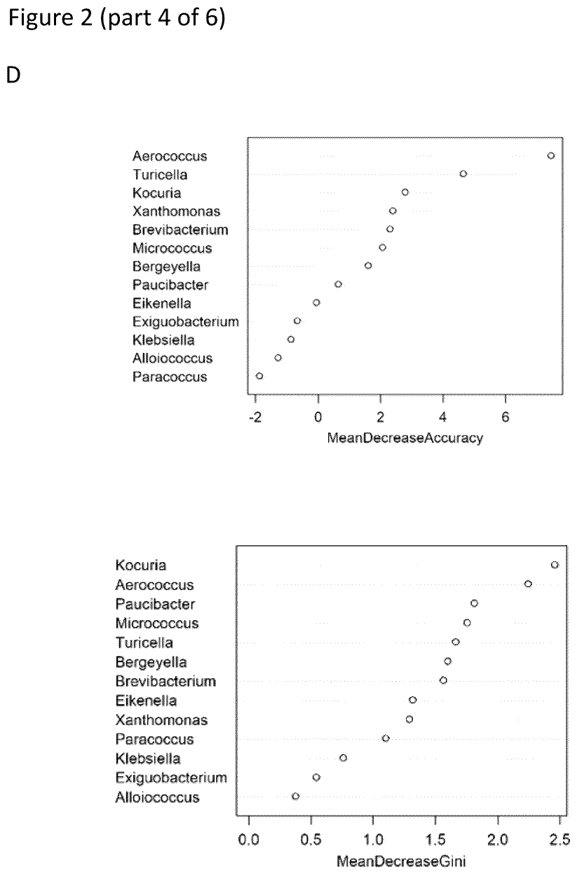

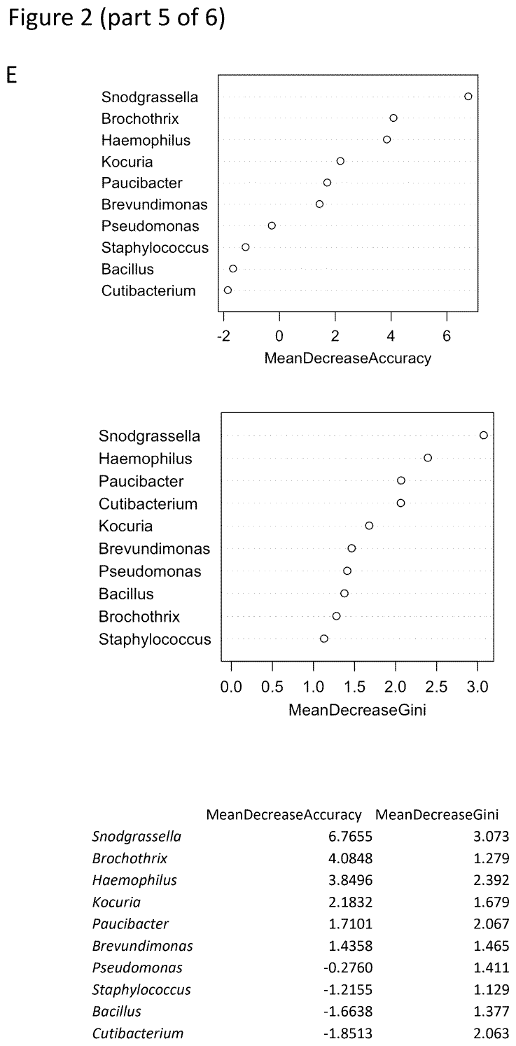

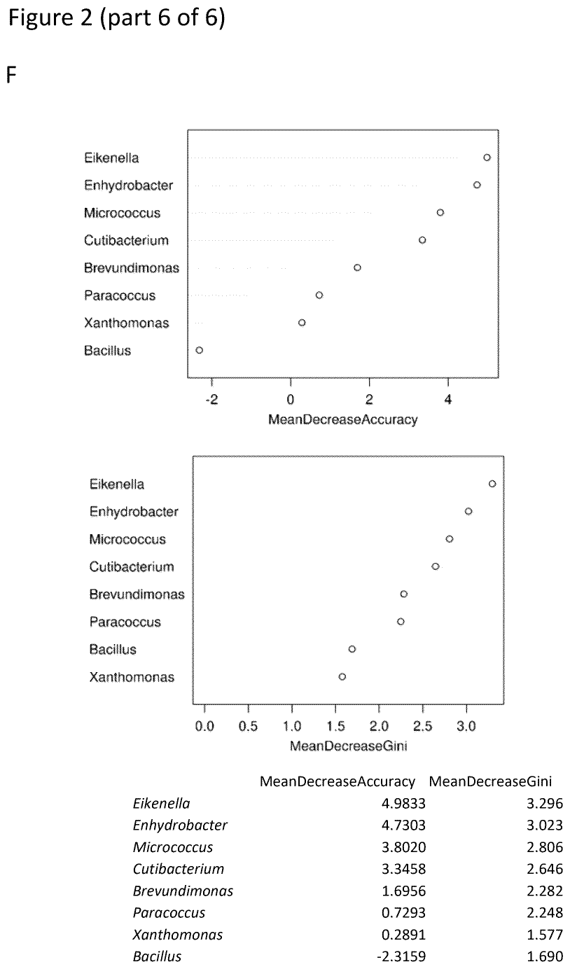

Bacteria identified with the foldchanges approach mentioned above are used to perform a Random Forest analysis. FIG. 2 shows relevant bacteria to define each skin type signatures in using Illumina MiSeq sequencing.

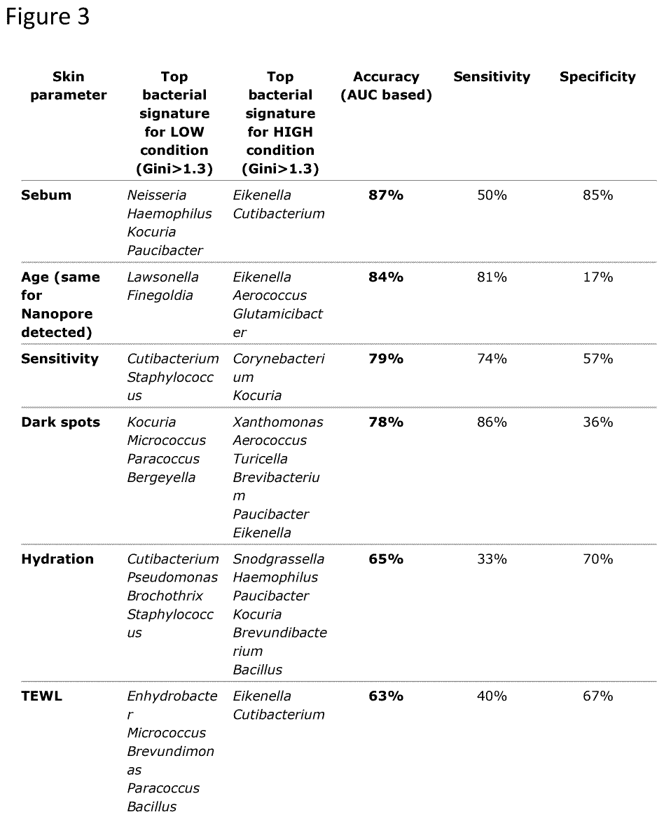

FIG. 3 shows the major relevant bacteria to define skin types in using Illumina MiSeq sequencing and the accuracy associated the signature of each skin parameter.

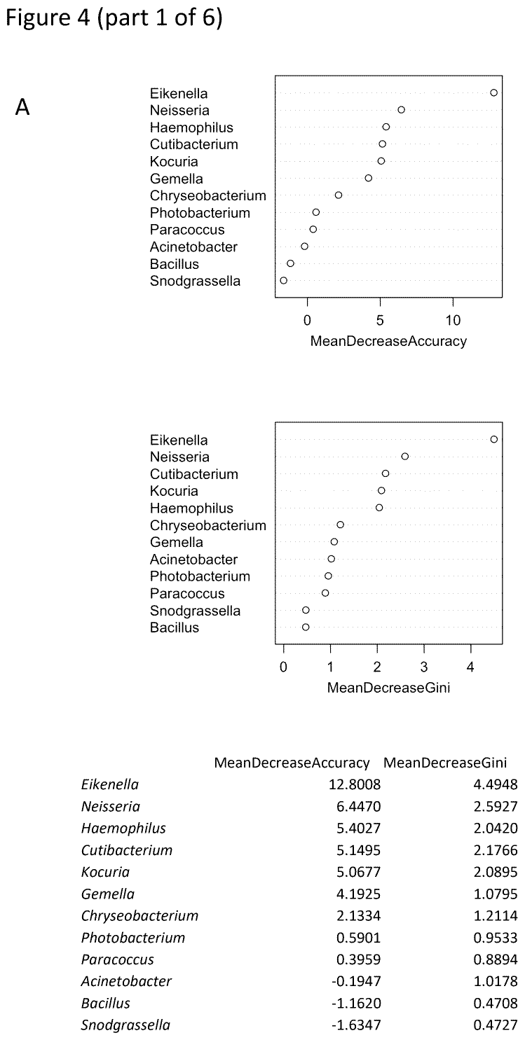

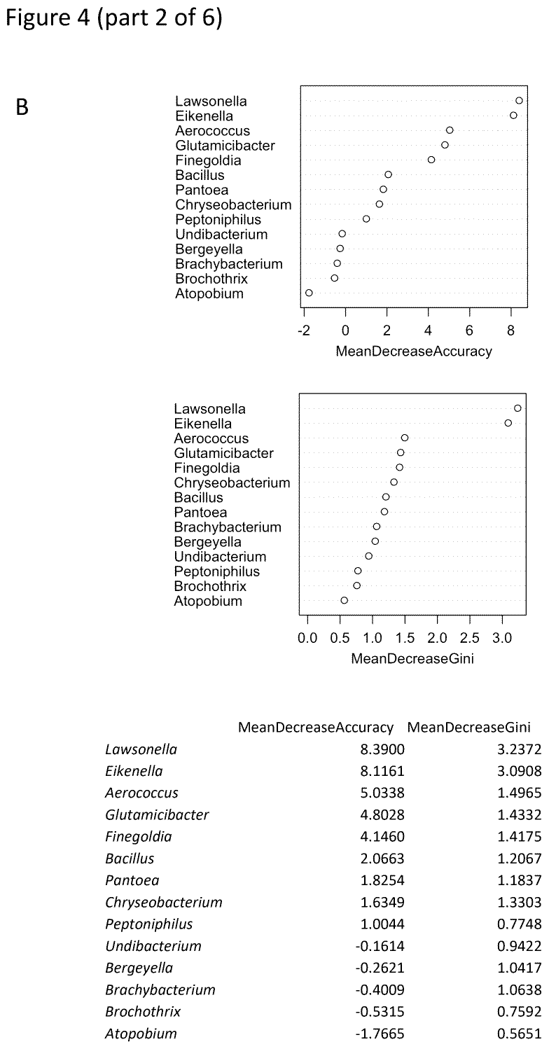

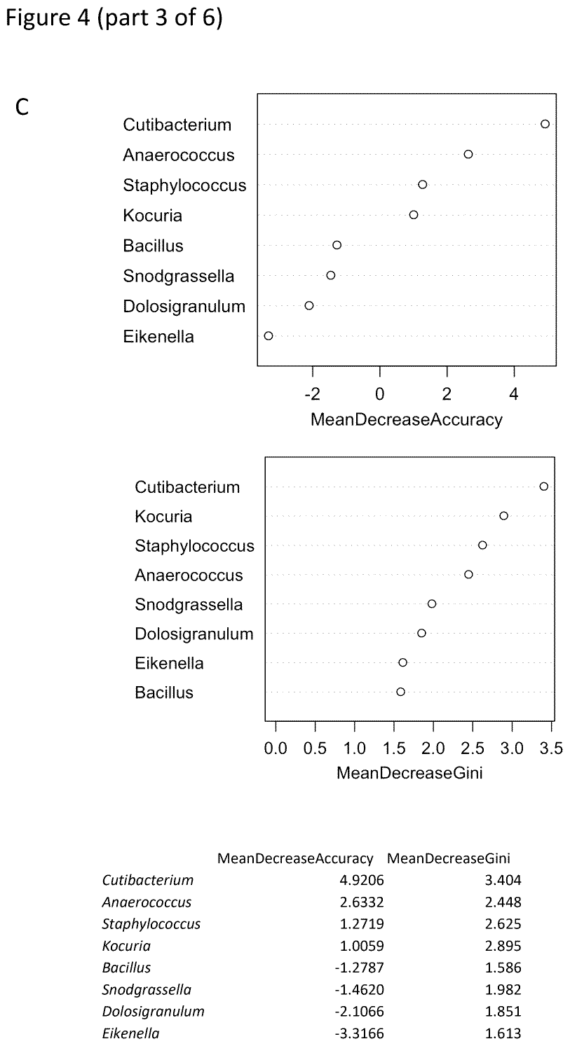

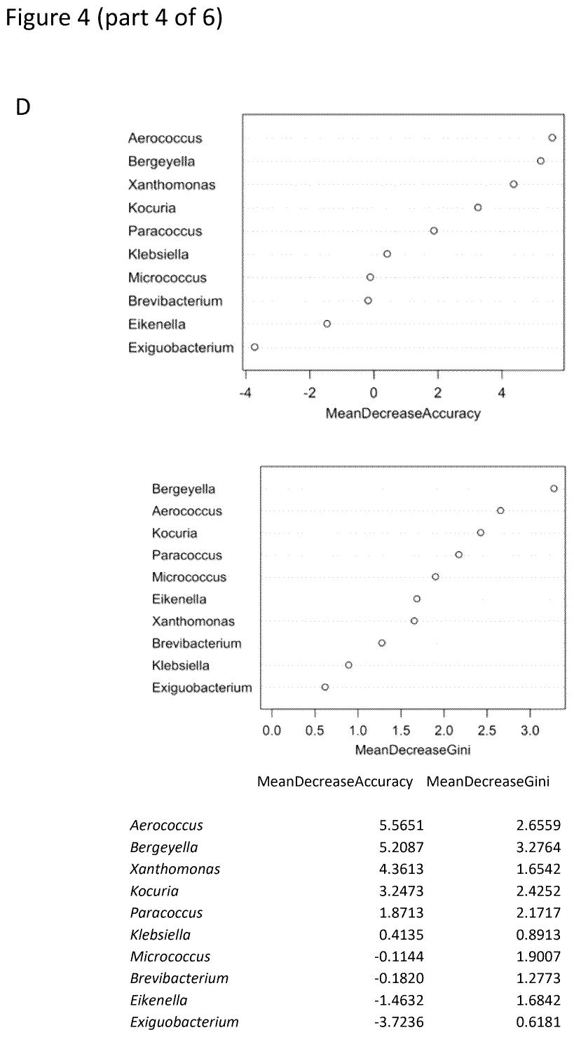

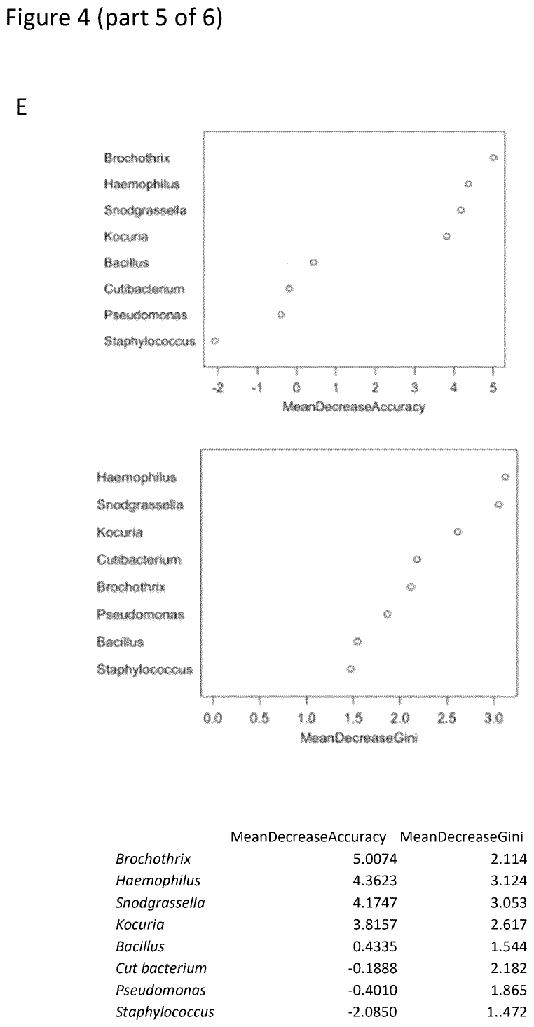

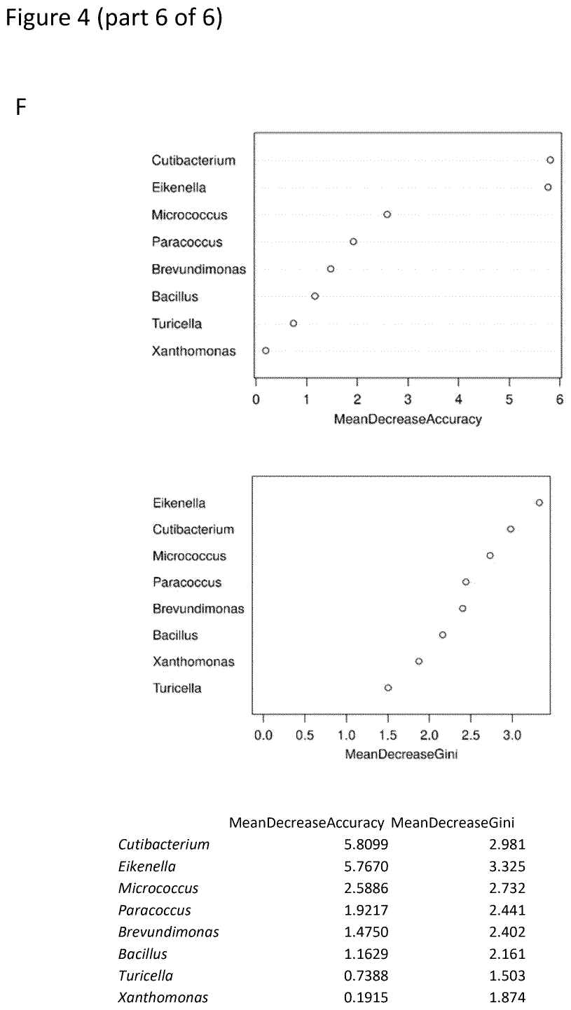

FIG. 4 shows relevant bacteria to define each skin type signatures in using Illumina MiSeq sequencing in limiting the signature to genera also detected with Nanopore sequencing technology; and FIG. 5 shows the relevant bacteria to define skin types and the accuracy associated the signature of each skin parameter in using Illumina MiSeq sequencing in limiting the signature to genera also detected with Nanopore sequencing technology.

### Example 3

Sampling, DNA preparation, sequencing and data treatment were as described in PCT application PCT/EP20201075530 and above, using the 16S full length approach.

Nanopore reads of the “pass” file, corresponding to reads with a good quality are assigned to bacterial taxa using a 16S database.

### Ratios Definition

These ratios include one or more bacteria typical of “LOW” skin parameter condition and one or more bacteria typical of a “HIGH” skin parameter condition. Different combination have been tried, relevance and selection have been assessed on the following parameters: 1/ bacteria considered must be detectable with Nanopore technology in a minimum of 3 samples (12 considered, detected in at least 25% of the samples), 2/ two skin parameters cannot be assessed using the same bacteria combination, 3/ bacteria used must provide a similar prediction on more than 80% of the samples between technologies (true correlation when LOW Illumina corresponds to a LOW Nanopore prediction and when HIGH Illumina corresponds to a HIGH Nanopore prediction defined. We accept a MIDDLE prediction in one of the technology as true whether if it corresponds to a MIDDLE, HIGH or LOW prediction in the other technology).

Ratio considered: (sum of % of selected typical bacteria of a “LOW” skin profile)/(sum of % of selected typical bacteria of a “HIGH” skin profile)

The definition of cut-offs (X values) have been predicted in comparing samples sequenced with both Illumina and Nanopore technology.

If the ratio is <X₁, the skin profile is considered as typical as “LOW”

If the ratio is >X₂, the skin profile is considered as typical as “HIGH”

If the ratio is >=X₁ and <=X₂, the skin profile is considered as “MEDIUM”

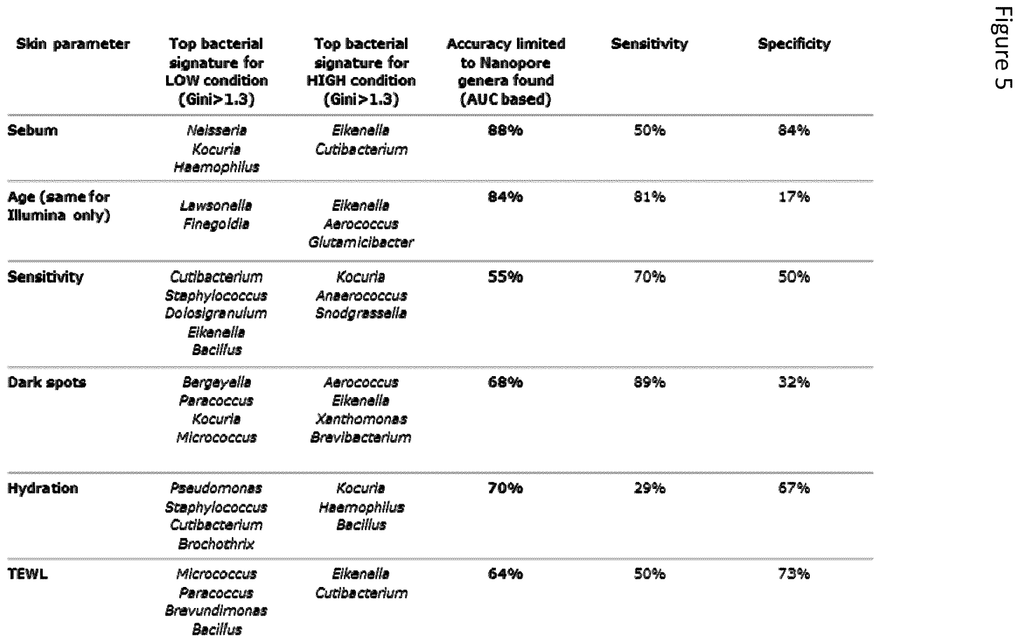

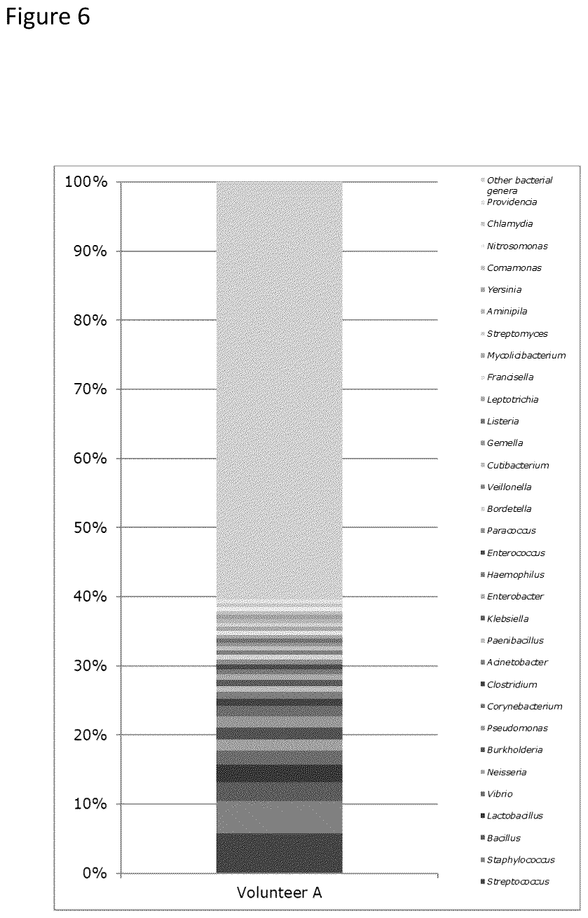

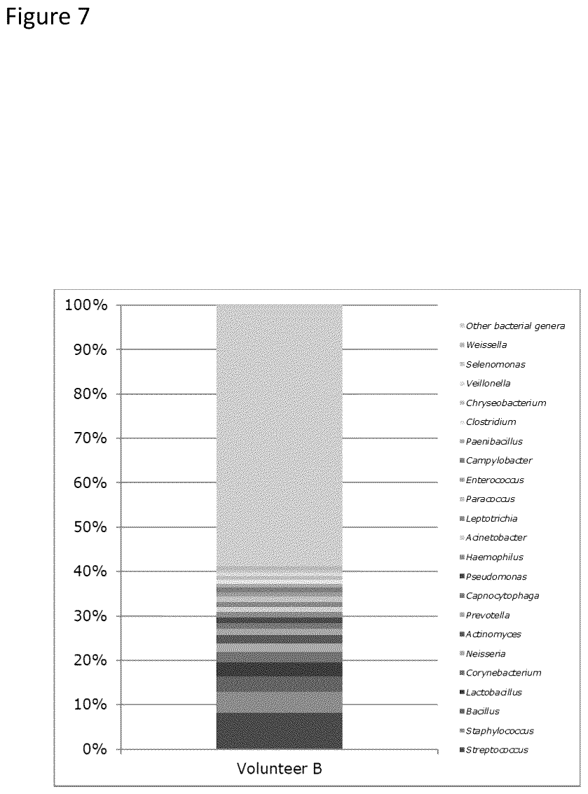

Taxonomy assignation of Nanopore reads of volunteer A is shown in FIG. 6, and taxonomy assignation of Nanopore reads of volunteer B is shown in FIG. 7.

### Volunteer A:

### Percentage of Bacteria of Interest:

| | Sebum level |
|---|---|
| Eikenella | 0.28 |
| Cutibacterium | 0.57 |
| Kocuria | 0.28 |
| Neisseria | 1.71 |
| Haemophilus | 0.71 |
| Ratio HIGH/LOW | 0.32 |
| | Age |
| Eikenella | 0.28 |
| Brochothrix | 0.00 |
| Lawsonella | 0.00 |
| Peptoniphilus | 0.43 |
| Finegoldia | 0.14 |
| Ratio HIGH/LOW | 0.5 |
| | Sensitivity |
| Corynebacterium | 1.57 |
| Kocuria | 0.28 |
| Snodgrassella | 0.14 |
| Cutibacterium | 0.57 |
| Ratio HIGH/LOW | 3.4 |
| | Spots level |
| Eikenella | 0.28 |
| Micrococcus | 0.14 |
| Paracoccus | 0.71 |
| Ratio HIGH/LOW | 0.33 |
| | Hydration |
| | level |
| Haemophilus | 0.71 |
| Snod | 0.14 |
| Brochothrix | 0.00 |
| Pseudomonas | 1.57 |
| Ratio HIGH/LOW | 0.55 |
| | TEWL level |
| Cutibacterium | 0.57 |
| Brevundimonas | 0.14 |
| Paracoccus | 0.14 |
| Ratio HIGH/LOW | 2.00 |

To define the skin type corresponding to this microbiota profile, ratios regarding bacterial specific of a skin type are used:

| | | Volunteer A | |
|---|---|---|---|
| | Cut-off values | score | Prediction |
| Sebum level | L < 0.28; H > 0.31 | 0.32 | HIGH |
| Age | L < 0.31; H > 0.5 | 0.50 | HIGH |
| Sensitivity | L < 0.75; H > 3.25 | 3.50 | HIGH |
| Spots level | L < 0.08; H > 0.27 | 0.33 | HIGH |
| Hydration level | L < 0.37; H > 0.5 | 0.55 | HIGH |
| TEWL level | L < 1.5; H > 2.6 | 2.00 | MIDDLE |

Using these ratios, adapted to each skin parameters, the skin profile of the volunteer A is defined as:
Sebum HIGH=Oily skin
Aged HIGH=Aged of more than 42 years old=Prematurely aged skin (because the volunteer is 41)
Sensitivity HIGH=Sensitive skin
Spots HIGH=Dark spot predisposed skin
Hydration HIGH=Well hydrated skin
TEWL MIDDLE=Normal skin barrier function

3/ Regarding these results, cosmetic recommendation can include 4 types of actives. Actives are listed with their commercial name followed by their INCI name. Actives with a proven effect on the microbiome are firstly listed.

### —To Reduce the Sebum Quantity:

Actives with a Proven Activity on the Sebum Regulation & on the Skin Microbiome:
Brightenyl® (Water (and) Glycerin (and) Diglucosyl Gallic Acid), Sopholiance™ S (Candida bombicola (and) Glucose (and) Methyl Rapeseedate Ferment (and) Water)
   Actives with a Proven Activity on the Sebum Regulation:
Bamboosilk; Erasyal™; K-phyto [PP] GHK; PrimalHyal™ 300; Safester™ A-75; Unireduce® R-35 Unisteron™ Y-50; Unitrienol™ T-27

### —To Improve the Age of the Skin:

Actives with a Proven Activity on the Ageing Signs & on the Skin Microbiome:
Agefinity™; Brightenyl®; Revivyl™; Synchronight™; Vetivyne™; Yogurtene® Balance
   Actives with a Proven Activity on Ageing Signs:
BlurHDR; Commipheroline®; Depollutine®; Easyliance® 2.0; Eau de Source Marine Eau Vitale™ d′Algue Bleue; Ellagi-CT; Hyalusphere®; Hydrintense™; Inoveol® CAFA Inoveol® EGCG; Inoveol® OLEU; Megassane®; Neodermyl™; NovHyal™ Biotech G; PrimalHyal 3K; PrimalHyal™ 50; PrimalHyal™ Gold; PrimalHyal™ Ultrafiller; Redens'In™; Rosmarinyl™; Rubixyl®; Softalia®; StimulHyal®; Tightenyl™; Unichondrin™ ATP; Unilactamin™ L-17; Unilucent™ HR-14; Unilucent™ PA-13; Uniprosyn® PS-18; Uniprotect® PT-3; Unirepair® T-43; Unisteron™ Y-50; Unisurrection™ S-61; Yogurtene®

### —To Improve the Sensitivity Status:

Actives with a Proven Activity on the Sensitive Status & on the Skin Microbiome:
Sensityl™
   Actives with a Proven Activity on the Sensitive Status:
Biogomm'age™ UE series; Biogomm'age™ WD series; BisaboLife™ (Bisabolol) D-Panthenyl Triacetate; Endothelyol™; Erasyal™; Grevilline™ PF; Inoveol® CAFA; Inoveol® EGCG; Inoveol® OLEU; Mariliance™; Neurophroline™; Ocâline™ Rosmarinyl™; Soothex®; Uniglucan™ G-51; Uniprotect® PT-3; Unirepair® T-43 Unisooth™ EG-28; Unisooth™ PN-47; Unisooth™ ST-32; Unispheres® Colour boosting Unispheres® Flashtone

### —To Improve Dark Spots Pre-Disposition:

Actives with a Proven Activity on the Dark Spot Modulation & on the Skin Microbiome:
Agefinity™; Brightenyl®
   Actives with a Proven Activity on the Dark Spot Modulation:
Axolight®; B-Lightyl™; Ellagi-CT; Megassane®; Neurophroline™; Unilucent™ HR-14; Unilucent™ PA-13 Uninontan™ U-34; Unispheres® Flashwhite; Wakamine™ Whitesphere™

### Volunteer B:

### Percentages of Bacteria of Interest:

| | Sebum level |
|---|---|
| Eikenella | 0.29 |
| Cutibacterium | 0.58 |
| Kocuria | 0.29 |
| Neisseria | 1.89 |
| Haemophilus | 1.16 |
| Ratio HIGH/LOW | 0.26 |
| | Age |
| Eikenella | 0.29 |
| Brochothrix | 0.00 |
| Lawsonella | 0.00 |
| Peptoniphilus | 0.29 |
| Finegoldia | 0.15 |
| Ratio HIGH/LOW | 0.67 |
| | Sensitivity |
| Corynebacterium | 2.47 |
| Kocuria | 0.29 |
| Snodgrassella | 0.15 |
| Cutibacterium | 0.58 |
| Ratio HIGH/LOW | 5 |
| | Spots level |
| Eikenella | 0.29 |
| Micrococcus | 0.29 |
| Paracoccus | 1.16 |
| Ratio HIGH/LOW | 0.20 |
| | Hydration level |
| Haemophilus | 1.16 |
| Snod | 0.15 |
| Brochothrix | 0.00 |
| Pseudomonas | 1.31 |
| Ratio HIGH/LOW | 1.00 |
| | TEWL level |
| Cutibacterium | 0.58 |
| Brevundimonas | 0.15 |
| Paracoccus | 0.58 |
| Ratio HIGH/LOW | 0.80 |

2/ To define the skin type corresponding to this microbiota profile, ratios regarding bacterial specific of a skin type are used.

| | Cut-off values | Volunteer B score | Prediction |
|---|---|---|---|
| Sebum level | L < 0.28; H > 0.31 | 0.26 | LOW |
| Age | L < 0.31; H > 0.5 | 0.67 | HIGH |
| Sensitivity | L < 0.75; H > 3.25 | 5.00 | HIGH |
| Spots level | L < 0.08; H > 0.27 | 0.20 | MIDDLE |
| Hydration level | L < 0.37; H > 0.5 | 1.00 | HIGH |
| TEWL level | L < 1.5; H > 2.6 | 0.80 | LOW |

Using these ratios, adapted to each skin parameters, the skin profile of the volunteer A is defined as:
Sebum LOW=Not oily skin
Aged HIGH=Aged of more than 42 years old=Prematurely aged skin (because the volunteer is 39)
Sensitivity HIGH=Sensitive skin
Spots MIDDLE=Not Dark spot predisposed skin
Hydration HIGH=Well hydrated skin
TEWL LOW=Low skin barrier function

3/ Regarding these results, cosmetic recommendation can include 3 types of actives:

### —To Improve the Age of the Skin:

Actives with a Proven Activity on the Ageing Signs & on the Skin Microbiome:
Agefinity™, Brightenyl®, Revivy|™, Synchronight™, Vetivyne™
Yogurtene® Balance
   Actives with a Proven Activity on Ageing Signs:
BlurHD® (Dipropylene Glycol (and) Gardenia Florida Fruit Extract)
Commipheroline® (Caprylic/Capric Triglyceride (and) Commiphora mukul Resin Extract)
Depollutine® (Water (and) Arginine PCA (and) Phaeodactylum tricornutum Extract)
Easyliance® 2.0 (Acacia Senegal Gum (and) Rhizobian Gum)
Eau de Source Marine (Spring Sea Water) (Sea water)
Eau Vitale™ d′Algue Bleue (Water (and) Spirulina Platensis Extract)
Ellagi-C™ (Propanediol (and) Anogeissus LeiocarpA Bark Extract)
Hyalusphere® (Sodium Hyaluronate Encapsulated)
Hydrintense™ (Sea Water (and) Propanediol (and) Porphyridium Cruentum Extract)
Inoveol® CAFA (Water (and) Caffeyl Glucoside)
Inoveol® EGCG (Water (and) Epigallocatechin Gallatyl Glucoside)
Inoveol® OLEU (Water (and) Oleuropeinyl Glucoside)
Megassane® (Caprylic/Capric Triglyceride (and) Phaeodactylum tricornutum Extract
Neodermyl™Gylcerin (and) Water (and) Methylglucoside Phosphate (and) Copper Lysinate/Prolinate)
NovHyal™ Biotech G (Glycerin (and) Water (and) Disodium Acetyl Glucosamine Phosphate)
PrimalHyal 3K (Hydrolyzed hyaluronic acid)
PrimalHyal™ 50 (Hydrolyzed Hyaluronic Acid)
PrimalHyal™ Gold (Water (and) PEG-8 Caprylic/Capric Glycerides (and) Sodium Hyaluronate (and) Octyldodeceth-25) PrimalHyal™ Ultrafiller (Sodium Acetylated Hyaluronate)
Redens'In™ (Commiphora mukul Resin Extract (and) Sodium Hyaluronate Encapsulated)
Rosmarinyl™ (Glucoside Water (and) Rosmarinyl Glucoside)
Rubixyl® (Water (and) Glycerin (and) Hexapeptide-48 HCL)
Softalia® (Fusanus Spicatus Kernel Oil)
StimulHyal® (Calcium Ketogluconate)
Tightenyl™ (Glycerin (and) Water (and) Disodium Acetyl Glucosamine Phosphate (and) Sodium Glucuronate (and) Magnesium Sulfate)
Unichondrin™ ATP (Water (and) Butylene Glycol (and) Hydrolyzed Vegetable Protein (and) Adenosine Triphosphate (and) Sodium Chondroitin Sulfate)
Unilactamin™ L-17 (Water (and) Butylene Glycol (and) Adenosine Triphosphate (and) Hydrolyzed Milk Protein (and) Niacinamide)
Unilucent™ HR-14 (Water (and) Haberlea Rhodopensis Leaf Extract)
Unilucent™ PA-13 (Panthenyl Triacetate (and) Acetyl Rheum rhaponticum Root Extract)
Uniprosyn® PS-18 (Water (and) Butylene Glycol (and) Niacinamide (and) Adenosine Triphosphate (and) Hydrolyzed Oat Protein)
Uniprotect® PT-3 (Panthenyl Triacetate (and) Ethyl Linoleate (and) Oleyl Alcohol (and) Tocopherol)
Unirepair® T-43 (Water (and) Butylene Glycol (and) Acetyl Tyrosine (and) Proline (and) Hydrolyzed Vegetable Protein (and) Adenosine Triphosphate)
Unisteron™ Y-50 (Oleyl Alcohol (and) Dioscorea villosa (Wild Yam) Root Extract (and) Hexyldecanol (and) Glycine soja (Soybean) Sterols)
Unisurrection™ S-61 (Water (and) Beta vulgaris (Beet) Root Extract (and) Glycerin (and) Haberlea Rhodopensis Leaf Extract (and) Yeast Extract)
Yogurtene® (Yogurt powder)

### —To Improve the Sensitivity Status:

Actives with a Proven Activity on the Sensitive Status & on the Skin Microbiome:
Sensityl™
   Actives with a Proven Activity on the Sensitive Status:
Biogomm'age™, Biogomm'age™, BisaboLife™, D-Panthenyl Triacetate, Endothelyol™, Erasyal™, Grevilline™ PF, Inoveol® CAFA, Inoveol® EGCG, Inoveol® OLEU, Mariliance™ Neurophroline™, Ocâline™, Rosmarinyl™, Soothex®, Uniglucan™ G-51, Uniprotect® PT-3 Unirepair® T-43, Unisooth™ EG-28, Unisooth™ PN-47, Unisooth™ ST-32 Unispheres® Colour boosting, Unispheres® Flashtone G
   —To Improve the Skin Barrier Function (the Recommendation would have been the Same in Case of LOW Hydration Profile):
   Actives with a Proven Activity on the Barrier Function & on the Skin Microbiome:
Agefinity™, Brightenyl® Sensityl™ Vetivyne™, Yogurtene® Balance
   Actives with a Proven Activity on the Barrier Function:
Abdoliance™, Appygreen™ 812, Axolight®, B-Lightyl, Bamboosilk, Biogomm'age™ UE series, Biogomm'age™ WD series, BisaboLife™, BlurHDR, Ceramide II/Uniblend™ 2G, Commipheroline® Cristalhyal® Range, D-Panthenyl Triacetate, Depollutine®, Easyliance® 2.0 Ellagi-C™, Endothelyol™, Erasyal™, Evercool®, Exfoliance Range, Glossyliance™, Grevilline™ PF Hairsphere, Hyalusphere®, Hydreïs™, Hydrintense™, Inoveol® CAFA, Inoveol® EGCG Inoveol® OLEU, K-phyto [SC] Camellia K-phyto [PP] GHK, Karanja Oil, Kendi oil, Lithocosmetics, Mariliance™, Megassane®, Muciliance® Neodermyl™, Neurophroline™, NovHyal™, Ocâline™, Pongamia Extract PrimalHyal 3K, PrimalHyal™ 300, PrimalHyal™, PrimalHyal™, Pro-DG™ Questice® Range, Questice Plus: Redensyl™, Redens'In™, ResistHyal™, Revivyl™ Rosmarinyl, Rubixyl®, Safester™ A-75, Sens'Hyal™, Silkalun™ Sinodor®, Softolive™, Soligel™, Soothex® Sophogreen™ Plus, Sopholiance™, StimulHyal®, Synchronight, Syner-GX™, Tightenyl™, Unichondrin™, Uniglucan™ G

### Example 4. Smoking Impacts Microbiota Composition of Smokers Forehead and Scalp

14 non-smokers/13 smokers: average age 32.1 (8.1) and 33.3 (9.0) respectively. Measure of 11 parameters in sin (hydratation, sebum, trans epidemial water loss, pH, wrinkles, brown spots, readness, porphyrin, visible spots).

4 swabbed areas (forehead, cheek, scalp). Sequencing of V3V4 subunit of 16S rRNA gene (using MiSeq) as described previously.

### Results:

Skin microbiota diversity is significantly impacted by smoking habits. Forehead ans scalp of smokers microbiota are significantly different from those of non-smokers (see table 1, table 2 and table 3). The skin microbiota modifications are close to the one observed with aging. A model was set to define smoking habits based on skin microbiota data.

TABLe 1
Cheek.

| | | | | Mean | |
|---|---|---|---|---|---|
| | | | | % for | Mean |
| | | | | non- | % for |
| Genus | foldchange | pvalue | padj | smokers | smokers |
| Micrococcus | −4.51 | 0.0002 | 0.0062 | 1.24 | 0.95 |
| Eikenella | 7.52 | 0.0105 | 0.1523 | 2.13 | 3.95 |
| Gemella | −2.19 | 0.0170 | 0.1645 | 0.81 | 0.44 |
| Corynebacterium | 2.67 | 0.0241 | 0.1745 | 4.49 | 11.07 |
| Finegoldia | 2.20 | 0.0394 | 0.1854 | 0.29 | 0.49 |

TABLE 2
Forehead

| | | | | Mean % | Mean |
|---|---|---|---|---|---|
| | | | | for non- | % for |
| Genus | foldchange | pvalue | padj | smokers | smokers |
| Micrococcus | −3.61 | 0.0052 | 0.1557 | 0.90 | 0.49 |
| Bacillus | −3.63 | 0.0216 | 0.3237 | 0.12 | 0.07 |
| Finegoldia | 2.29 | 0.0454 | 0.4540 | 0.09 | 0.43 |

TABLE 3
Scalp

| | | | | Mean % | Mean |
|---|---|---|---|---|---|
| | | | | for non- | % for |
| Genus | foldchange | pvalue | padj | smokers | smokers |
| Corynebacterium | 5.46 | 0.0007 | 0.0189 | 0.18 | 3.68 |
| Anaerococcus | 5.18 | 0.0026 | 0.0333 | 0.01 | 0.18 |
| Finegoldia | 4.37 | 0.0113 | 0.0983 | 0.01 | 0.07 |
| Cutibacterium | −3.50 | 0.0218 | 0.1416 | 63.63 | 48.12 |
| Micrococcus | 3.96 | 0.0421 | 0.2187 | 0.09 | 0.14 |

1. A method of predicting whether a subject has a high level of a first skin characteristic, a low level of the first skin characteristic, or an intermediate level of the first skin characteristic in a human subject wherein the method comprises:
a) providing a skin microbial sample taken from a subject and generating a test microbial profile from the skin microbial sample;
b) comparing the test microbial profile with a microbial signature that is predictive of a high level of the first characteristic or a low level of the first characteristic; and
c) determining whether the test microbial profile is that of skin with a high level of the first characteristic, a low level of the first characteristic, or an intermediate level of the first characteristic.

2. The method according to claim 1 wherein generating the test microbial profile comprises:
ii) detecting the presence of and/or determining the abundance of and/or calculating the percentage abundance of each genera of a microbial signature that is predictive of a high level of the first skin characteristic or a low level of the first skin characteristic; optionally
iii) summing the percentage abundance or abundance of each genera that is predictive of a high level of the first characteristic; and summing the percentage abundance of each genera that is predictive of a low level of the first characteristic; optionally
iv) calculating the ratio of the sum of the percentage abundance of each genera that is predictive of the high level of the first characteristic to the sum of the percentage abundance of each genera that is predictive of a low level of the first characteristic to generate a test ratio.

3. The method according to claim 2 wherein the method comprises:
v) comparing the test ratio of (iv) to a pre-defined upper threshold ratio and a pre-defined lower threshold ratio, wherein a test ratio that is greater than the upper threshold ratio predicts that the subject has skin with a high level of the first characteristic; a test ratio that is lower than the lower threshold ratio predicts that the subject has skin with a low level of the first characteristic; and a test ratio between the upper and lower threshold ratios predicts an intermediate skin characteristic; and/or
vi) comparing the presence, abundance or percentage abundance of the genera predictive of a high level of a low level of the characteristic determined in (ii) or sum of the abundance or percentage abundance of (iii) to a microbial signature.

4. The method according to claim 1, wherein the microbial signature that is predictive of a high level of a first characteristic comprises any one of or more of the following genera:
Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and Xanthomonas;
wherein the microbial signature that is predictive of a low level of a first characteristic comprises any one or more of the following genera:
Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and Xanthomonas.

5. (canceled)

6. The method according to claim 1, wherein:
the high level of a characteristic is a high level of aged skin and the low level of a characteristic is a low level of aged skin;
the high level of a characteristic is a high level of brown spots and the low level of a characteristic is a low level of brown spots;
the high level of a characteristic is a high level of sensitivity and the low level of a characteristic is a low level of sensitivity;
the high level of a characteristic is a high level of sebum and the low level of a characteristic is a low level of sebum;
the high level of a characteristic is a high level of hydration and the low level of a characteristic is a low level of hydration
the high level of a characteristic is a high level of aged skin linked to smoking and the low level of a characteristic is a low level of aged skin linked to smoking; and/or
the high level of a characteristic is a high level of barrier function and the low level of a characteristic is a low level of barrier function.

7. The method according to claim 1, wherein the high level of a first characteristic is a high level of age and the low level of the first characteristic is a low level of age, and
wherein the genera predictive of a high level of age comprises any one or more or all of:
a) Eikenella and/or Brochothrix;
b) Eikenella, Aerococcus and/or Glutamicibacter;
c) Aerococcus, Atopobium, Bacillus, Brachybacterium, Brochothrix, Chryseobacterium, Eikenella, Eubacterium, Glutamicibacter and/or Pantoea; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;
wherein the genera predictive of a low level of age comprises any one or more or all of:
a) Finegoldia, Lawsonella and/or Peptoniphilus;
b) Finegoldia and/or Lawsonella;
c) Bergeyella, Cloacibacterium, Finegoldia, Lawsonella, Peptoniphilus and/or Undibacterium; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

8. (canceled)

9. The method according to claim 1, wherein the high level of a first characteristic is a high level of age and the low level of the first characteristic is a low level of age, and
a) wherein the genera predictive of a high level of age comprises any one or more or all of: Eikenella and/or Brochothrix,
and wherein the genera predictive of a low level of age comprises any one or more or all of: Finegoldia, Lawsonella and/or Peptoniphilus,
b) wherein the genera predictive of a high level of age comprises any one or more or all of: Eikenella, Aerococcus and/or Glutamicibacter,
and wherein the genera predictive of a low level of age comprises any one or more or all of: Finegoldia and/or Lawsonella,
c) wherein the genera predictive of a high level of age comprises any one or more or all of:
Aerococcus, Atopobium, Bacillus, Brachybacterium, Brochothrix, Chryseobacterium, Eikenella, Eubacterium, Glutamicibacter and/or Pantoea;
and wherein the genera predictive of a low level of age comprises any one or more or all of:
Bergeyella, Cloacibacterium, Finegoldia, Lawsonella, Peptoniphilus and/or Undibacterium.

10. The method according to claim 1, wherein the high level of a first characteristic is a high level of brown spots and the low level of the first characteristic is a low level of brown spots, and
wherein the genera predictive of high brown spots comprises any one or more or all of:
a) Eikenella;
b) Eikenella, Aerococcus, Xanthomonas and/or Brevibacterium;
c) Paucibacter, Turicella, Xanthomonas, Brevibacterium, Eikenella and/or Aerococcus;
d) Aerococcus, Brevibacterium, Eikenella, Klebsiella, Paucibacter, Turicella and/or Xanthomonas; and/or
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;
wherein the genera predictive of low brown spots comprises any one or more or all of:
a) Micrococcus and/or Paracoccus;
b) Kocuria, Micrococcus, Paracoccus and/or Bergeyella;
c) Alloiococcus, Bergeyella, Exiguobacterium, Kocuria, Micrococcus and/or Paracoccus; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

11. (canceled)

12. The method according to claim 1, wherein the high level of a first characteristic is a high level of brown spots and the low level of the first characteristic is a low level of brown spots, and
a) wherein the genera predictive of a high level of brown spots comprises any one or more or all of: Eikenella,
and wherein the genera predictive of a low level of brown spots comprises any one or more or all of: Micrococcus and/or Paracoccus;
b) wherein the genera predictive of a high level of brown spots comprises any one or more or all of: Eikenella, Aerococcus, Xanthomonas, and/or Brevibacterium
and wherein the genera predictive of a low level of brown spots comprises any one or more or all of: Kocuria, Micrococcus, Paracoccus and/or Bergeyella;
c) wherein the genera predictive of a high level of brown spots comprises any one or more or all of: Paucibacter, Turicella, Xanthomonas, Brevibacterium, Eikenella, and/or Aerococcus
and wherein the genera predictive of a low level of brown spots comprise any one or more or all of: Kocuria, Micrococcus, Paracoccus and/or Bergeyella
d) wherein the genera predictive of a high level of brown spots comprises any one or more or all of: Aerococcus, Brevibacterium, Eikenella, Klebsiella, Paucibacter, Turicella and/or Xanthomonas
and wherein the genera predictive of a low level of brown spots comprise any one or more or all of: Alloiococcus, Bergeyella, Exiguobacterium, Kocuria, Micrococcus and/or Paracoccus.

13. The method according to claim 1, wherein the high level of the first characteristic is a high level of sebum and the low level of the first characteristic is a low level of sebum, and wherein the genera predictive of high sebum comprises any one or more or all of:
a) Cutibacterium and/or Eikenella;
b) Actinobacillus, Anaerococcus, Bacillus, Cutibacterium, Eikenella, Peptoniphilus, Snodgrassella, Staphylococcus and/or Turicella; and/or
c) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;
wherein the genera predictive of a low level of sebum comprises any one or more or all of:
a) Haemophilus, Kocuria and/or Neisseria;
b) Haemophilus, Kocuria, Neisseria and/or Paucibacter;
c) Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and/or Brochothix;
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

14. (canceled)

15. The method according to claim 1, wherein the high level of the first characteristic is a high level of sebum and the low level of the first characteristic is a low level of sebum, and
a) wherein the genera predictive of high sebum comprises any one or more or all of:
Cutibacterium and/or Eikenella
and wherein the genera predictive of low sebum comprise any one or more or all of:
Haemophilus, Kocuria, Neisseria.
b) wherein the genera predictive of high sebum comprises any one or more or all of: Cutibacterium and/or Eikenella
and wherein the genera predictive of low sebum comprise any one or more or all of:
Haemophilus, Kocuria, Neisseria, Paucibacter;
c) wherein the genera predictive of high sebum comprises any one or more or all of:
Actinobacillus, Anaerococcus, Bacillus, Cutibacterium, Eikenella, Peptoniphilus, Snodgrassella, Staphylococcus and/or Turicella;
and wherein the genera predictive of low sebum comprises any one or more or all of:
Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus,
d) wherein the genera predictive of a high level of sebum comprises any one or more or all of:
Actinobacillus, Anaerococcus, Bacillus, Cutibacterium, Eikenella, Peptoniphilus, Snodgrassella, Staphylococcus and/or Turicella;
and wherein the genera predictive of a low level of sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and/or Brochothix.

16. The method according to claim 1, wherein the high level of a first characteristic is a high level of sensitivity and the low level of the first characteristic is a low level of sensitivity, and
wherein the genera predictive of a high level of sensitivity comprises any one or more or all of:
a) Corynebacterium and/or Kocuria;
b) Anaerococcus, Kocuria and/or Snodgrassella;
c) Actinobacillus, Anaerococcus, Corynebacterium, Filifactor, Kocuria, Roseomonas and/or Snodgrassella; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;
wherein the genera predictive of a low level of sensitivity comprises any one or more or all of:
a) Cutibacterium;
b) Cutibacterium and/or Staphylococcus;
c) Bacillus, Cutibacterium, Dolosigranulum, Eikenella, Staphylococcus and/or Stenotrophomonas;
d) Bacillus, Campylobacter, Cutibacterium, Delftia, Dolosigranulum, Eikenella, Frigoribacterium, Glutamicibacter, Moraxella, Serratia, Staphylococcus, Stenotrophomonas and/or Undibacterium;
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

17. (canceled)

18. The method according to claim 1, wherein the high level of a first characteristic is a high level of sensitivity and the low level of the first characteristic is a low level of sensitivity, and
a) wherein the genera predictive of a high level of sensitivity comprises any one or more or all of: Corynebacterium and/or Kocuria;
and wherein the genera predictive of a low level of sensitivity comprise any one or more or all of: Cutibacterium.
b) wherein the genera predictive of a high level of sensitivity comprises any one or more or all of: Anaerococcus, Kocuria and/or Snodgrassella.
and wherein the genera predictive of a low level of sensitivity comprise any one or more or all of:
Bacillus, Cutibacterium, Dolosigranulum, Eikenella, Staphylococcus and/or Stenotrophomonas
c) wherein the genera predictive of a high level of sensitivity comprises any one or more or all of: Corynebacterium and/or Kocuria
and wherein the genera predictive of a low level of sensitivity comprises any one or more or all of: Cutibacterium and/or Staphylococcus;
d) wherein the genera predictive of a high level of sensitivity comprises any one or more or all of: Actinobacillus, Anaerococcus, Corynebacterium, Filifactor, Kocuria, Roseomonas and/or Snodgrassella;
and wherein the genera predictive of a low level of sensitivity comprises any one or more or all of: Bacillus, Campylobacter, Cutibacterium, Delftia, Dolosigranulum, Eikenella, Frigoribacterium, Glutamicibacter, Moraxella, Serratia, Staphylococcus, Stenotrophomonas and/or Undibacterium.

19. The method according to claim 1, wherein the high level of a first characteristic is a high level of hydration and the low level of the first characteristic is a low level of hydration, and
wherein the genera predictive of a high level of hydration comprises any one or more or all of:
a) Haemophilus and/or Snodgrassella;
b) Bacillus, Haemophilus and/or Kocuria;
c) Bacillus, Haemophilus, Kocuria, Paucibacter and/or Snodgrassella;
d) Allorhizobium, Amaricoccus, Bacillus, Brevundimonas, Falsirhodobacter, Haemophilus, Kocuria, Lautropia, Paucibacter and/or Snodgrassella; and/or
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;
wherein the genera predictive of a low level of hydration comprises any one or more or all of:
a) Brochothrix and/or Pseudomonas;
b) Brochothrix, Cutibacterium, Pseudomonas and/or Staphylococcus;
c) Brochothrix, Cutibacterium, Pseudomonas, Serratia, Staphylococcus, Turicella and/or Undibacterium; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

20. (canceled)

21. The method according to claim 1, wherein the high level of a first characteristic is a high level of hydration and the low level of the first characteristic is a low level of hydration, and
a) wherein the genera predictive of a high level of hydration comprises any one or more or all of: Haemophilus and/or Snodgrassella,
and wherein the genera predictive of a low level of hydration comprises any one or more or all of: Brochothrix and/or Pseudomonas;
b) wherein the genera predictive of a high level of hydration comprises any one or more or all of: Bacillus, Haemophilus and/or Kocuria,
and wherein the genera predictive of a low level of hydration comprises any one or more or all of: Brochothrix, Cutibacterium, Pseudomonas and/or Staphylococcus
c) wherein the genera predictive of a high level of hydration comprises any one or more or all of: Bacillus, Haemophilus, Kocuria, Paucibacter and/or Snodgrassella
and wherein the genera predictive of low hydration comprises any one or more or all of: Brochothrix, Cutibacterium, Pseudomonas and/or Staphylococcus
d) wherein the genera predictive of a high level of hydration comprises any one or more or all of: Allorhizobium, Amaricoccus, Bacillus, Brevundimonas, Falsirhodobacter, Haemophilus, Kocuria, Lautropia, Paucibacter and/or Snodgrassella;
and wherein the genera predictive of a low level of hydration comprises any one or more or all of: Brochothrix, Cutibacterium, Pseudomonas, Serratia, Staphylococcus, Turicella and/or Undibacterium.

22. The method according to claim 1, wherein the high level of a first characteristic is a high level of barrier function age and the low level of the first characteristic is a low level of barrier function, and
wherein the genera predictive of a low level of barrier function comprises any one or more or all of:
a) Cutibacterium;
b) Eikenella and/or Cutibacterium;
c) Brochothrix, Cutibacterium, Eikenella, Lactobacillus, Turicella and/or Veillonella; and/or
d) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas;
wherein the genera predictive of a high level of barrier function comprises any one or more or all of:
a) Paracoccus and/or Brevundimonas;
b) Micrococcus, Paracoccus, Brevundimonas, Bacillus and/or Xanthomonas;
c) Enhydrobacter, Micrococcus, Brevundimonas, Paracoccus and/or Bacillus;
d) Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and/or Xanthomonas; and/or
e) Acinetobacter, Actinobacillus, Aerococcus, Alloiococcus, Allorhizobium, Amaricoccus, Anaerococcus, Atopobium, Bacillus, Bergeyella, Brachybacterium, Brevibacterium, Brevundimonas, Brochothrix, Campylobacter, Chryseobacterium, Cloacibacterium, Corynebacterium, Cutibacterium, Delftia, Dolosigranulum, Eikenella, Enhydrobacter, Eubacterium, Exiguobacterium, Falsirhodobacter, Filifactor, Finegoldia, Frigoribacterium, Fusobacterium, Gemella, Glutamicibacter, Granulicatella, Haematobacter, Haemophilus, Klebsiella, Kocuria, Lachnoanaerobaculum, Lactobacillus, Lactococcus, Lautropia, Lawsonella, Leptotrichia, Micrococcus, Moraxella, Neisseria, Pantoea, Paracoccus, Paucibacter, Peptoniphilus, Photobacterium, Porphyromonas, Pseudomonas, Roseomonas, Serratia, Snodgrassella, Staphylococcus, Stenotrophomonas, Streptococcus, Turicella, Undibacterium, Veillonella and/or Xanthomonas.

23. (canceled)

24. The method according to claim 1, wherein the high level of a first characteristic is a high level of barrier function and the low level of the first characteristic is a low level of barrier function, and
a) wherein the genera predictive of a low level of barrier function comprises any one or more or all of: Cutibacterium
and wherein the genera predictive of a high level of barrier function comprises any one or more or all of: Paracoccus and/or Brevundimonas
b) wherein the genera predictive of a low level of barrier function comprises any one or more or all of: Eikenella and/or Cutibacterium
and wherein the genera predictive of a high level of barrier function comprises any one or more or all of: Micrococcus, Paracoccus, Brevundimonas, Bacillus and/or Xanthomonas
c) wherein the genera predictive of a low level of barrier function comprises any one or more or all of: Eikenella and/or Cutibacterium
and wherein the genera predictive of a high level of barrier function comprises any one or more or all of: Enhydrobacter, Micrococcus, Brevundimonas, Paracoccus and/or Bacillus
d) wherein the genera predictive of a low level of barrier function comprises any one or more or all of: Brochothrix, Cutibacterium, Eikenella, Lactobacillus, Turicella and/or Veillonella and wherein the genera predictive of a high level of barrier function comprises any one or more or all of: Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and/or Xanthomonas.

25. The method according to claim 1, wherein the high level of a first characteristic is a high level of aged skin linked to smoking and the low level of the first characteristic is a low level of aged skin linked to smoking, wherein the microbiota is measured in the cheek, the forehead and/or the scalp, and
wherein the genera predictive of a high level of aged skin linked to smoking comprises:
a) Micrococcus, Bacillus, Eikenella, Gemella, Corynebacterium, Finegoldia, Cutibacterium and/or Anaerococcus.

26. (canceled)

27. The method according to claim 1, wherein said generating a test microbial profile in step (a) involves sequencing a region of the microbial genome.

28. (canceled)

29. (canceled)

30. (canceled)

31. (canceled)

32. (canceled)

33. A method for categorising skin type into each of the following characteristics:
a) a low level of sensitivity, a high level of sensitivity, or an intermediate level of sensitivity;
b) a low level of sebum, a high level of sebum, or an intermediate level of sebum;
c) a low level of hydration, a high level of hydration, or an intermediate level of hydration;
d) a low level of brown spots, a high level of brown spots, or an intermediate level of brown spots;
e) a low level of aged skin, a high level of aged skin, or an intermediate level of aged skin;
f) a low level of barrier function, a high level of barrier function, or an intermediate level of barrier function;
wherein the method comprises determining the percentage abundance, abundance, or presence of bacteria from each of the following genera in a skin microbial sample:
Brevundimonas, Brochothrix, Corynebacterium, Cutibacterium, Eikenella, Finegoldia, Haemophilus, Kocuria, Lawsonella, Micrococcus, Neisseria, Paracoccus, Peptoniphilus, Pseudomonas and Snodgrassella.

34. The method of categorising skin type into a low level of age, a high level of age, or an intermediate level of age of claim 33, wherein the method comprises determining the percentage abundance, abundance, or presence of bacteria from each of the following genera in a skin microbial sample:
a) Eikenella, Brochothrix Finegoldia, Lawsonella and Peptoniphilus; optionally wherein
the genera predictive of a high level of age comprises any one or more or all of: Eikenella and/or Brochothrix; and
the genera predictive of a low level of age comprises any one or more or all of: Finegoldia, Lawsonella and/or Peptoniphilus
b) Eikenella, Aerococcus, Glutamicibacter Finegoldia and Lawsonella; optionally wherein
the genera predictive of a high level of age comprises any one or more or all of: Eikenella, Aerococcus and/or Glutamicibacter; and
the genera predictive of a low level of age comprises any one or more or all of: Finegoldia and/or Lawsonella;
c) Aerococcus, Atopobium, Bacillus, Brachybacterium, Brochothrix, Chryseobacterium, Eikenella, Eubacterium, Glutamicibacter Pantoea, Bergeyella, Cloacibacterium, Finegoldia, Lawsonella, Peptoniphilus and Undibacterium; optionally wherein
the genera predictive of a high level of age comprises any one or more or all of: Aerococcus, Atopobium, Bacillus, Brachybacterium, Brochothrix, Chryseobacterium, Eikenella, Eubacterium, Glutamicibacter and/or Pantoea; and
the genera predictive of a low level of age comprises any one or more or all of: Bergeyella, Cloacibacterium, Finegoldia, Lawsonella, Peptoniphilus and/or Undibacterium.

35. The method of categorising skin type into a low level of brown spots, a high level of brown spots, or an intermediate level of brown spots of claim 33, wherein the method comprises determining the percentage abundance, abundance, or presence of bacteria from each of the following genera in a skin microbial sample:
a) Eikenella, Micrococcus and Paracoccus; optionally wherein
the genera predictive of a high level of brown spots comprises any one or more or all of: Eikenella; and
the genera predictive of a low level of brown spots comprises any one or more or all of: Micrococcus and/or Paracoccus;
b) Eikenella, Aerococcus, Xanthomonas, Brevibacterium, Kocuria, Micrococcus, Paracoccus and Bergeyella; optionally wherein
the genera predictive of a high level of brown spots comprises any one or more or all of:
Eikenella, Aerococcus, Xanthomonas, and/or Brevibacterium; and
the genera predictive of a low level of brown spots comprises any one or more or all of: Kocuria, Micrococcus, Paracoccus and/or Bergeyella;
c) Paucibacter, Turicella, Xanthomonas, Brevibacterium, Eikenella, Aerococcus, Kocuria, Micrococcus, Paracoccus and Bergeyella; optionally wherein
the genera predictive of a high level of brown spots comprises any one or more or all of: Paucibacter, Turicella, Xanthomonas, Brevibacterium, Eikenella, and/or Aerococcus; and
the genera predictive of a low level of brown spots comprise any one or more or all of: Kocuria, Micrococcus, Paracoccus and/or Bergeyella;
d) Aerococcus, Brevibacterium, Eikenella, Klebsiella, Paucibacter, Turicella Xanthomonas, Alloiococcus, Bergeyella, Exiguobacterium, Kocuria, Micrococcus and Paracoccus; optionally wherein
the genera predictive of a high level of brown spots comprises any one or more or all of:
Aerococcus, Brevibacterium, Eikenella, Klebsiella, Paucibacter, Turicella and/or Xanthomonas; and
the genera predictive of a low level of brown spots comprise any one or more or all of: Alloiococcus, Bergeyella, Exiguobacterium, Kocuria, Micrococcus and/or Paracoccus.

36. The method of categorising skin type into a low level of sensitivity, a high level of sensitivity, or an intermediate level of sensitivity of claim 33, wherein the method comprises determining the percentage abundance, abundance, or presence of bacteria from each of the following genera in a skin microbial sample:
a) Corynebacterium, Kocuria and Cutibacterium; optionally wherein
the genera predictive of a high level of sensitivity comprises any one or more or all of Corynebacterium and/or Kocuria; and
the genera predictive of a low level of sensitivity comprise Cutibacterium;
b) Anaerococcus, Kocuria, Snodgrassella, Bacillus, Cutibacterium, Dolosigranulum, Eikenella, Staphylococcus and Stenotrophomonas; optionally wherein
the genera predictive of a high level of sensitivity comprises any one or more or all of:
Anaerococcus, Kocuria and/or Snodgrassella; and
the genera predictive of a low level of sensitivity comprise any one or more or all of: Bacillus, Cutibacterium, Dolosigranulum, Eikenella, Staphylococcus and/or Stenotrophomonas;
c) Corynebacterium, Kocuria, Cutibacterium and Staphylococcus; optionally wherein
the genera predictive of a high level of sensitivity comprises any one or more or all of: Corynebacterium and/or Kocuria; and
the genera predictive of a low level of sensitivity comprises any one or more or all of:
Cutibacterium and/or Staphylococcus;
d) Actinobacillus, Anaerococcus, Corynebacterium, Filifactor, Kocuria, Roseomonas Snodgrassella, Bacillus, Campylobacter, Cutibacterium, Delftia, Dolosigranulum, Eikenella, Frigoribacterium, Glutamicibacter, Moraxella, Serratia, Staphylococcus, Stenotrophomonas and Undibacterium; optionally wherein
the genera predictive of a high level of sensitivity comprises any one or more or all of: Actinobacillus, Anaerococcus, Corynebacterium, Filifactor, Kocuria, Roseomonas and/or Snodgrassella; and
the genera predictive of a low level of sensitivity comprises any one or more or all of:
Bacillus, Campylobacter, Cutibacterium, Delftia, Dolosigranulum, Eikenella, Frigoribacterium, Glutamicibacter, Moraxella, Serratia, Staphylococcus, Stenotrophomonas and Undibacterium;

37. The method of categorising skin type into a low level of sebum, a high level of sebum, or an intermediate level of sebum of claim 33, wherein the method comprises determining the percentage abundance, abundance, or presence of bacteria from each of the following genera in a skin microbial sample:
a) Cutibacterium, Eikenella Haemophilus, Kocuria and Neisseria; optionally wherein
the genera predictive of high sebum comprises any one or more or all of: Cutibacterium and/or Eikenella; and
the genera predictive of low sebum comprise any one or more or all of: Haemophilus, Kocuria, Neisseria;
b) Cutibacterium, Eikenella, Haemophilus, Kocuria, Neisseria and Paucibacter; optionally wherein
the genera predictive of high sebum comprises any one or more or all of: Cutibacterium and/or Eikenella; and
the genera predictive of low sebum comprise any one or more or all of: Haemophilus, Kocuria, Neisseria, Paucibacter;
c) Actinobacillus, Anaerococcus, Bacillus, Cutibacterium, Eikenella, Peptoniphilus, Snodgrassella, Staphylococcus Turicella, Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, and Streptococcus; optionally wherein
the genera predictive of high sebum comprises any one or more or all of: Actinobacillus, Anaerococcus, Bacillus, Cutibacterium, Eikenella, Peptoniphilus, Snodgrassella, Staphylococcus and/or Turicella; and
the genera predictive of low sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus;
d) Actinobacillus, Anaerococcus, Bacillus, Cutibacterium, Eikenella, Peptoniphilus, Snodgrassella, Staphylococcus, Turicella Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and Brochothix; optionally wherein
the genera predictive of a high level of sebum comprises any one or more or all of: Actinobacillus, Anaerococcus, Bacillus, Cutibacterium, Eikenella, Peptoniphilus, Snodgrassella, Staphylococcus and/or Turicella; and
the genera predictive of a low level of sebum comprises any one or more or all of: Acinetobacter, Brachybacterium, Brevibacterium, Brevundimonas, Chryseobacterium, Fusobacterium, Gemella, Haematobacter, Haemophilus, Kocuria, Lachnoanaerobaculum, Lactococcus, Leptotrichia, Neisseria, Paracoccus, Paucibacter, Photobacterium, Porphyromonas, Roseomonas, Streptococcus and/or Brochothix.

38. The method of categorising skin type into a low level of hydration, a high level of hydration, or an intermediate level of hydration of claim 33, wherein the method comprises determining the percentage abundance, abundance, or presence of bacteria from each of the following genera in a skin microbial sample:
a) Haemophilus, Snodgrassella, Brochothrix and Pseudomonas; optionally wherein
the genera predictive of a high level of hydration comprises any one or more or all of: Haemophilus and/or Snodgrassella; and
the genera predictive of a low level of hydration comprises any one or more or all of: Brochothrix and/or Pseudomonas;
b) Bacillus, Haemophilus, Kocuria, Brochothrix, Cutibacterium, Pseudomonas and Staphylococcus; optionally wherein
the genera predictive of a high level of hydration comprises any one or more or all of:
Bacillus, Haemophilus and/or Kocuria; and
the genera predictive of a low level of hydration comprises any one or more or all of: Brochothrix, Cutibacterium, Pseudomonas and/or Staphylococcus;
c) Bacillus, Haemophilus, Kocuria, Paucibacter and/or Snodgrassella; optionally wherein
the genera predictive of a high level of hydration comprises any one or more or all of: Bacillus, Haemophilus, Kocuria, Paucibacter, Snodgrassella, Brochothrix, Cutibacterium, Pseudomonas and Staphylococcus; and
the genera predictive of low hydration comprises any one or more or all of: Brochothrix, Cutibacterium, Pseudomonas and/or Staphylococcus;
d) Allorhizobium, Amaricoccus, Bacillus, Brevundimonas, Falsirhodobacter, Haemophilus, Kocuria, Lautropia, Paucibacter, Snodgrassella; Brochothrix, Cutibacterium, Pseudomonas, Serratia, Staphylococcus, Turicella and Undibacterium; optionally wherein
the genera predictive of a high level of hydration comprises any one or more or all of:
Allorhizobium, Amaricoccus, Bacillus, Brevundimonas, Falsirhodobacter, Haemophilus, Kocuria, Lautropia, Paucibacter and/or Snodgrassella; and
the genera predictive of a low level of hydration comprises any one or more or all of: Brochothrix, Cutibacterium, Pseudomonas, Serratia, Staphylococcus, Turicella and/or Undibacterium.

39. The method of categorising skin type into a low level of barrier function, a high level of barrier function, or an intermediate level of barrier function of claim 33, wherein the method comprises determining the percentage abundance, abundance, or presence of bacteria from each of the following genera in a skin microbial sample:
a) Cutibacterium, Paracoccus and Brevundimonas; optionally wherein
the genera predictive of a low level of barrier function comprises any one or more or all of: Cutibacterium; and
the genera predictive of a high level of barrier function comprises any one or more or all of: Paracoccus and/or Brevundimonas;
b) Eikenella, Cutibacterium, Micrococcus, Paracoccus, Brevundimonas, Bacillus and Xanthomonas; optionally wherein
the genera predictive of a low level of barrier function comprises any one or more or all of: Eikenella and/or Cutibacterium; and
the genera predictive of a high level of barrier function comprises any one or more or all of:
Micrococcus, Paracoccus, Brevundimonas, Bacillus and/or Xanthomonas;
c) Eikenella, Cutibacterium Enhydrobacter, Micrococcus, Brevundimonas, Paracoccus and Bacillus; optionally wherein
the genera predictive of a low level of barrier function comprises any one or more or all of: Eikenella and/or Cutibacterium; and
the genera predictive of a high level of barrier function comprises any one or more or all of:
Enhydrobacter, Micrococcus, Brevundimonas, Paracoccus and/or Bacillus;
d) Brochothrix, Cutibacterium, Eikenella, Lactobacillus, Turicella, Veillonella Aerococcus, Allorhizobium, Amaricoccus, Bacillus, Brevundimonas, Chryseobacterium, Enhydrobacter, Filifactor, Granulicatella, Klebsiella, Micrococcus, Paracoccus, Paucibacter and Xanthomonas; optionally wherein
the genera predictive of a low level of barrier function comprises any one or more or all of:
Brochothrix, Cutibacterium, Eikenella, Lactobacillus, Turicella and/or Veillonella; and
the genera predictive of a high level of barrier function comprises any one or more or all of: Allorhizobium, Amaricoccus, Aerococcus, Bacillus, Brevundimonas, Chryseobacterium, Enhydrobacter, Filifactor, Granulicatella, Klebsiella, Micrococcus, Paracoccus, Paucibacter and/or Xanthomonas.

40. The method of categorising skin type into a low level of aged skin linked to smoking, a high level of aged skin linked to smoking, or an intermediate level of aged skin linked to smoking of claim 33, wherein the method comprises determining the percentage abundance, abundance, or presence of bacteria from each of the following genera in a skin microbial sample:
a) Micrococcus, Bacillus, Eikenella, Gemella, Corynebacterium, Finegoldia, Cutibacterium and/or Anaerococcus.

41. (canceled)

42. (canceled)

43. The method according to claim 33, wherein the method comprises determining the ratio of total abundance of all genera associated with a high level of the characteristic to the total abundance of all genera associated with a low level of the characteristic.

44. A method of identifying a microbial signature predictive of either a high level of a first characteristic or a low level of the same first characteristic wherein the method comprises:
a) (i) providing a number of control skin microbial samples wherein the samples are taken from more than one subject showing a high level of the characteristic, generating a microbial profile from the skin microbial samples, and optionally calculating the percentage abundance of each genera in each sample; and
b) (ii) providing a number of control skin microbial samples wherein the samples are taken from more than one subject showing a low level of the characteristic, generating a microbial profile from the skin microbial samples, and optionally calculating the percentage abundance of each genera in each sample; and
optionally
c) generating a mean percentage abundance of each genera from the control samples showing a high level of the characteristic; and
d) generating a mean percentage abundance of each genera from the control samples showing a low level of the characteristic; and
e) identifying genera of microbes that are more prevalent in skin showing the high level of the characteristic; and
f) identifying genera of microbes that are more prevalent in skin showing the low level of the characteristic;
and optionally
g)
i) for the control skin samples showing a high level of the characteristic, summing the mean percentage abundance of each genera identified in (e) and summing the mean percentage abundance of each genera identified in (f); and
ii) calculating the ratio of the sum of the mean percentage abundance of each genera identified in (e) to the sum of the mean percentage abundance of each genera identified in (f) giving an upper threshold ratio; and
iii) for the control skin samples showing a low level of the characteristic, summing the mean percentage abundance of each genera identified in (e) and summing the mean percentage abundance of each genera identified in (f); and
iv) calculating the ratio of the sum of the mean percentage abundance of each genera identified in (e) to the sum of the mean percentage abundance of each genera identified in (f) giving a lower threshold
wherein:
a test sample having a test ratio of greater than the upper threshold ratio is considered to be a sample that has a high level of the characteristic;
a test sample having a test ratio of lower than the lower threshold ratio is considered to be a sample that has a low level of the characteristic; and
a test sample having a test ratio of between the upper threshold ratio and the lower threshold ratio is considered to be a sample that has an intermediate level of the characteristic; and/or
h) wherein a test sample comprising genera of microbes that are more prevalent in skin showing a high level of the characteristic is considered to predict that the skin has a high level of the characteristic, and wherein a test sample comprising genera of microbes that are more prevalent in skin showing a low level of the characteristic is considered to predict that the skin has a low level of the characteristic.

45. The method according to claim 44 wherein the:
the high level of a characteristic is a high level of sensitivity and the low level of a characteristic is a low level of sensitivity;
the high level of a characteristic is a high level of sebum and the low level of a characteristic is a low level of sebum;
the high level of a characteristic is a high level of hydration and the low level of a characteristic is a low level of hydration;
the high level of a characteristic is a high level of brown spots and the low level of a characteristic is a low level of brown spots;
the high level of a characteristic is a high level of aged skin and the low level of a characteristic is a low level of aged skin; and/or
the high level of a characteristic is a high level of barrier function and the low level of a characteristic is a low level of barrier function.

46. The method according to claim 44, wherein the method comprises the following steps:
species or genera are discarded from further analysis if they were not identified in more than 10%, 15%, 20%, 25%, 30%, 35%, 40%, 45%, 50%, 55%, 60%, 65%, 70%, 75% or 80% of control samples taken from subjects with a high level of the characteristic; and/or
species or genera are discarded from further analysis if they were not identified in more than 10%, 15%, 20%, 25%, 30%, 35%, 40%, 45%, 50%, 55%, 60%, 65%, 70%, 75% or 80% of control samples taken from subjects with a low level of the characteristic.

47. The method according to claim 44, wherein the method comprises identifying a first microbial signature predictive of a high level of a first skin characteristic or a low level of a first skin characteristic, and a second microbial signature predictive of a high level of a second skin characteristic or a low level of a second characteristic, the first and the second microbial signatures are different.

48. A method of cosmetic treatment, comprising predicting whether a subject has a high level of a first skin characteristic or a low level of the first skin characteristic according to the method of claim 1, and treating the skin with a cosmetic selected to improve the condition of the first skin characteristic.

49. A method of monitoring the efficacy of a cosmetic treatment, comprising:
(i) providing a first test skin microbial sample taken from a subject and predicting whether the subject has a high level of at least a first skin characteristic, a low level of at least a first skin characteristic, or an intermediate level of at least a first skin characteristic according to the method of claim 1;
(ii) treating the skin with a cosmetic selected to modulate the level of the at least one characteristic; and
(iii) providing a second test skin microbial sample taken from a subject and predicting whether the subject has a high level of at least a first skin characteristic, a low level of at least a first skin characteristic, or an intermediate level of at least a first skin characteristic according to the method of claim 1;
(iv) determining whether the level of the at least a first skin characteristic has been desirably modulated.

50. (canceled)

51. (canceled)

52. (canceled)

53. (canceled)


SEQUENCE LISTING (4 sequences)

SEQ ID NO: 1 - DNA, 36 bp, Artificial Sequence
  Forward 16S primer; where “n” = a or c or g or t/u
atcgcctaccgtgacnagagtttgatcmtggctcag

SEQ ID NO: 2 - DNA, 36 bp, Artificial Sequence
  Reverse 16S primer; where “n” = a or c or g or t/u
atcgcctaccgtgacncggttaccttgttacgactt

SEQ ID NO: 3 - DNA, 17 bp, Artificial Sequence
  16S-Mi341F forward primer; where “n” = a or c or g or t/u
cctacgggnggcwgcag

SEQ ID NO: 4 - DNA, 21 bp, Artificial Sequence
  16S-Mi805R reverse primer
gactachvgggtatctaatcc